(19) United States
(12) Patent Application Publication
(10) Pub. No.: US 2022/0226355 A1
(43) Pub. Date: Jul. 21, 2022

(54) TIMED ALTERNATE ADMINISTRATION OF DECITABINE AND 5-AZACYTIDINE FOR CANCER TREATMENT

(71) Applicant: The Cleveland Clinic Foundation, Cleveland, OH (US)

(72) Inventors: Yogen Saunthararajah, Cleveland Heights, OH (US); Vamsidhar Velcheti, Scarsdale, NY (US); Kai Kang, Cleveland, OH (US); Xiaorong Gu, Cleveland, OH (US); Rita Tohme, Cleveland, OH (US)

(21) Appl. No.: 17/613,709

(22) PCT Filed: May 22, 2020
(86) PCT No.: PCT/US2020/034321

§ 371 Date: Nov. 23, 2021

Related U.S. Application Data
(60) Provisional application No. 62/852,807, filed on May 24, 2019.

(51) Int. Cl. A61K 31/706; A61P 35/00; A61K 31/7072; A61K 38/19; G01N 33/50

(52) U.S. Cl. CPC A61K 31/706; A61P 35/00; G01N 33/5094; A61K 38/193; A61K 31/7072

(57) ABSTRACT

Provided herein are compositions, systems, kits, and methods for treating a patient with cancer by alternate administration of decitabine and 5-azacytidine, or administration of decitabine two times per week on consecutive days, which is generally timed to bypass auto-dampening and exploit cross-priming. Such administration is combined with an inhibitor of the enzyme cytidine deaminase (e.g., tetrahydrouridine) that otherwise rapidly catabolizes decitabine and 5-azacytidine. In certain embodiments, the time between cycles of decitabine and 5-azacytidine administration is about three to four days or five to ten days.

The present application claims priority to U.S. Provisional application 62/852,807, filed May 24, 2019, which is herein incorporated by reference in its entirety.

### FIELD

Provided herein are compositions, systems, kits, and methods for treating a patient with cancer by alternate administration of decitabine and 5-azacytidine, or administration of decitabine two times per week on consecutive days, which is generally timed to bypass auto-dampening and exploit cross-priming. Such administration is combined with an inhibitor of the enzyme cytidine deaminase (e.g., tetrahydrouridine) that otherwise rapidly catabolizes decitabine and 5-azacytidine. In certain embodiments, the time between cycles of decitabine and 5-azacytidine administration is about three to four days or five to ten days.

### BACKGROUND

Lung cancer is the leading cause of cancer mortality world-wide, claiming ˜1.7 million lives per year, more than liver and colorectal cancers combined. A landmark recent advance was approval of immune checkpoint blockade (ICB) using anti-PD-1/PD-L1 monoclonal antibodies to treat metastatic lung cancer. Unfortunately, only ˜20% of patients treated this way have durable benefits (1). New and rational complementary therapies are thus needed.

Lung cancers contain a high mutational burden that should require broader dependence on immune checkpoints for immune evasion than response rates to ICB thus far indicate (2). To be recognized by T-cells, however, antigenic peptides must be processed by proteasomes and chaperoned to the cancer cell surface for presentation by major histocompatibility complex (MHC) molecules. Any step in this antigen presentation process can be downregulated by repressive epigenetic changes, e.g., DNA methylation by DNA methyltransferase 1 (DNMT1) (reviewed in (3-5)). Accordingly, in several pre-clinical cancer models, DNMT1-depletion by the pyrimidine nucleoside analogs decitabine or 5-azacytidine increased expression of MHC molecules and associated immune co-stimulatory molecules, promoted tumor infiltration by IFN-γ producing and cytotoxic T-cells, and augmented anti-tumor effects of ICB (3-5). Transcription repression by DNMT1 and DNA methylation moreover silences other tumor suppressor genes in cancer cells, e.g., epithelial-differentiation genes which would otherwise antagonize the master regulator of cell growth and division MYC and terminate cancer cell self-renewal (reviewed in (6)). That is, DNMT1-depletion has been shown to not just augment immune-recognition of tumors, and hence enhance efficacy of immune checkpoint inhibitors, but also directly cytoreduces cancers, by a non-cytotoxic (non-apoptosis based) pathway that operates even in p53-null cancers refractory to apoptosis-based, and immune-disrupting, chemotherapy and radiation (6).

Overall, however, clinical results with decitabine or 5-azacytidine to treat solid tumor malignancies, alone or in combination with ICB, have not been as impressive as predicted by pre-clinical data (reviewed in (7)). One reason became evident in a clinical trial of intravenously-infused decitabine in patients with thoracic malignancies. Despite sustaining decitabine plasma concentrations of >40 nM for ˜72 hours, enough to cause grade 3/4 myelosuppression, molecular pharmacodynamic effects of DNA hypomethylation in lung cancer tissues were evident in <25% of patients (8). This indicated that levels of active drug in lung cancer tissues in vivo were minimal despite drug accumulation to excessive, cytotoxic levels in bone marrow. A reason for such uneven tissue effects was suggested by subsequent experiments showing that the catabolic enzyme cytidine deaminase (CDA), that deaminates decitabine and 5-azacytidine into non-DNMT1-depleting metabolites within minutes, is expressed unevenly across tissues (9-12), being highly expressed in many solid tissues (e.g., liver) but less so in others (e.g., bone marrow). Accordingly, decitabine was ineffective in a pre-clinical model of cancer localized to the liver, but co-administration of lower doses of decitabine with an inhibitor of CDA, tetrahydrouridine (THU), eliminated cancer cells in this CDA-rich organ sanctuary, importantly, without bone marrow cytotoxicity (9). Also noteworthy is that CDA levels are in general much higher in humans than in mice (13), a feature possibly contributing to general discrepancies between murine and human studies.

### SUMMARY

Provided herein are compositions, systems, kits, and methods for treating a patient with cancer by alternate administration of decitabine and 5-azacytidine, or administration of decitabine two times per week on consecutive days, which is generally timed to bypass auto-dampening and exploit cross-priming. Such administration is combined with an inhibitor of the enzyme cytidine deaminase (e.g., tetrahydrouridine) that otherwise rapidly catabolizes decitabine and 5-azacytidine. In certain embodiments, the time between cycles of decitabine and 5-azacytidine administration is about three to four days. or five to ten days.

In some embodiments, provided herein are methods of treating a human subject with cancer comprising, or consisting essentially of: a) administering a cytidine deaminase inhibitor to a human subject at a first time, wherein the human subject has cancer; b) administering decitabine to the subject at a second time, wherein the second time is between 0.5 and 7 hours after the first time; c) administering a cytidine deaminase inhibitor to the subject at a third time, wherein the third time is between 60 and 105 hours, or 106-240 hours, after the first time; and d) administering 5-azacytidine to the subject at a fourth time, wherein the fourth time is: i) 0.5 and 7 hours after the third time, and ii) between 60 and 105 hours, or 106-240 hours, after the second time. In certain embodiments, the methods further comprise: e) repeating steps a)-d) at least once, wherein the administering in step a) is conducted between 60 and 105 hours, or 106-240 hours, after the fourth time.

In certain embodiments, provided herein are methods of treating a human subject with cancer comprising, or consisting essentially of: a) administering 5-15 mg/kg of a cytidine deaminase inhibitor to a human subject at a first time, wherein the human subject has cancer; b) administering 0.07-0.4 mg/kg of decitabine to the subject at a second time, wherein the second time is between 0.5 and 7 hours after the first time; c) administering 5-15 mg/kg of a cytidine deaminase inhibitor to the subject at a third time, wherein the third time is between 60 and 105 hours, or 106-240 hours, after the first time; and d) administering 0.5-4 mg/kg of 5-azacytidine to the subject at a fourth time, wherein the fourth time is: i) 0.5 and 7 hours after the third time, and ii) between 60 and 105 hours, or 106-240 hours, after the second time. In certain embodiments, the methods further comprise repeating steps a)-d) at least once, wherein the administering in step a) is conducted between 60 and 105 hours, or 106-240 hours, after the fourth time.

In particular embodiments, the subject does not receive any decitabine or 5-azacytidine except at the times specified in steps b) and d) respectively. In other embodiments, the repeating steps a)-d) at least once comprises repeating steps a)-d) at last five times (e.g., 5, 6, 7, 8, 9, 10, or 11 times). In further embodiments, the repeating steps a)-d) at least once comprises repeating steps a)-d) at last twelve times (e.g., 12, 13, 14, 15, 16, 17, 18 . . . 25 . . . or 50 times).

In particular embodiments, the cancer is selected from the group consisting of: pancreatic cancer, breast cancer, myeloid cancers, lymphoid cancers (e.g., T-cell lymphoid cancers), small cell lung cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In further embodiments, the administering 5-15 mg/kg of the cytidine deaminase inhibitor in steps a) and c) comprises administering 9-11 mg/kg of the cytidine deaminase inhibitor. In other embodiments, the administering 0.07-0.4 mg/kg of decitabine comprises administering 0.1-0.3 mg/kg of the decitabine. In other embodiments, the administering 0.07-0.4 mg/kg of decitabine comprises administering 0.15-0.17 mg/kg of the decitabine. In further embodiments, the administering 0.5-4 mg/kg of 5-azacytidine comprises administering 1-3 mg/kg of the 5-azacytidine. In other embodiments, the administering 0.5-4 mg/kg of 5-azacytidine comprises administering 1.5-1.7 mg/kg of the 5-azacytidine.

In some embodiments, the between 0.5 and 7 hours in step b) is between 1 and 5 hours (e.g., 1, 2, 3, 4, or 5 hours). In other embodiments, the between 0.5 and 7 hours in step d) is between 1 and 5 hours (e.g., 1, 2, 3, 4, or 5 hours). In particular embodiments, the between 60 and 105 hours, or 106-240 hours, in step d) is between 70 and 98 hours or between 72 and 96 hours (e.g., 72 . . . 80 . . . 88 . . . 96 hours) or between 150 and 175 hours (e.g., 150 . . . 155 . . . 170 . . . 175). In other embodiments, the between 60 and 105 hours, or 106-240 hours, in step c) is between 65 and 100 hours (e.g., 65 . . . 75 . . . 85 . . . 95 . . . and 100 hours), or between 150 and 175 hours (e.g., 150 . . . 155 . . . 170 . . . 175).

In certain embodiments, the methods further comprise: administering the subject a composition comprising human granulocyte colony-stimulating factor (GCSF). In other embodiments, the methods further comprise: testing a sample from the subject to determine absolute neutrophil count (ANC). In additional embodiments, the subject's ANC is below 1.5×10⁹, and the method further comprises administering the subject a composition comprising human granulocyte colony-stimulating factor (GCSF). In other embodiments, the subject does not receive any cytidine deaminase inhibitor except at the times specified in steps a) and c). In further embodiments, the cytidine deaminase inhibitor comprises tetrahydrouridine. In certain embodiments, the methods further comprise administering said subject an immune checkpoint inhibitor (e.g., anti-Pd1 antibody or biologically active fragment thereof).

In some embodiments, provided herein are methods of treating a human subject with cancer comprising, or consisting essentially of: a) administering decitabine to the subject on day one; b) administering decitabine to the subject on day two, wherein day two is the day immediately after the day one; c) administering decitabine to the subject on day eight, wherein the day eight is seven days after the day one, and wherein the subject does not receive any decitabine between the administering in step b) and the administering in step c); and d) administering decitabine to the subject on day nine, wherein the day nine is eight days after the day one.

In certain embodiments, the methods further comprise: repeating steps c) and d) at least once, wherein the subject does not receive any decitabine expect during the repeated administration of steps c) and d). In other embodiments, steps c) and d) are repeated at least twice (e.g., 2 times, 3 times, 4 times, 5 times, 6 times, 7 times, 8 times, 9 time, or 10 times). In additional embodiments, the methods further comprise administering a cytidine deaminase inhibitor to the subject on the same days as when the decitabine is administered. In further embodiments, the cytidine deaminase inhibitor is administered at a dosage of 5-15 mg/kg. In further embodiments, each administration of the decitabine is administered at dosage of 0.07-0.4 mg/kg.

### DESCRIPTION OF THE FIGURES

The patent or application file contains at least one drawing executed in color. Copies of this patent or patent application publication with color drawings will be provided by the Office upon request and payment of the necessary fee.

FIG. 1. Expression levels of key pyrimidine metabolism enzymes determine sensitivity of diverse cancer cell lines to decitabine (Dec) and 5-azacytidine (5Aza). A) Dec sensitivity correlates with DCK expression. Unbiased correlation of expression levels of pyrimidine metabolism enzymes and transporters (n=45) vs decitabine sensitivity (concentration of drug producing 50% growth inhibition at day 6 [GI50]) in the NC160 panel of cancer cell lines (n=60), public GI50 data, National Cancer Institute. Microarray gene expression data from GSE5846(47). Spearman correlation coefficients/p-values by SASv9. p-values <0.05 highlighted in green. B) Three-way plot of Dec GI50 vs DCK expression and doubling time. Cell line histologies: leukemia=blue, non-small cell lung=red, colon=green, central nervous system=gold, melanoma=grey, ovarian=pink, renal=light blue, prostate=black, breast=violet. C) Dec and 5Aza are converted into the DNMT1-depleting nucleotide by separate channels (green). Also shown are off-target effects (red) that disrupt nucleotide balances (yellow background). D) 5Aza sensitivity correlates with UCK2 expression. E) Three-way plot of 5Aza GI50 vs UCK2 expression and doubling time.

FIG. 2. The pyrimidine metabolism network reacts to Dec and 5Aza perturbation with adaptive shifts in enzyme expression that decrease nucleoside uptake. F1339 p53-null cancer cells were seeded at D0 and treated with DMSO, deoxycytidine (DC, 0.5 μM), cytidine (CY, 5 μM), Dec (0.5 μM) and 5Aza (5 μM). A) Dec has been shown to inhibit TYMS (17, 48) and 5Aza to inhibit ribonucleotide reductase (RRM1) (21), disrupting cellular nucleotide balances in specific ways. The enzyme expression shifts dampen uptake of the instigating drug (auto-resistance) but prime for uptake of the sister drug (cross-priming). CDA, that is upregulated by Dec, inactivates both Dec and 5Aza, and can be inhibited by tetrahydrouridine (THU). B) Dec and 5Aza, but not the natural nucleosides, slowed cell growth. Cell counts by automated counter, mean±SD. C) Dec increased UCK2 and decreased DCK, while 5Aza did the opposite. Gene expression by Q-PCR, median±interquartile range (IQR) (3 biological replicates), *p<0.05, **p<0.01. 2-sided Mann-Whitney test. D) Protein expression tracked the mRNA expression changes. Western blot.

FIG. 3. Non-cytotoxic concentrations of Dec and 5Aza suppress chemorefractory p53-null SCLC cells. A) High doses of etoposide and cisplatin were ineffective in vivo. B6/129 F1 mice were inoculated via tail vein with F1339-luc SCLC cells (0.3×10⁶ cells/mouse). After documentation of lung invasion by live-imaging, mice were distributed to PBS or combination cisplatin 12 mg/kg intraperitoneal (IP) 1×/week+etoposide 8 mg/kg IP 3×/week (n=5/group). B) No benefit of cisplatin+etoposide. Mice were euthanized for signs of distress. C) Dec and 5Aza, but not cyarabine at a concentration equimolar to the Dec, was anti-proliferative to p53-null SCLC cells. Dec and cytarabine 0.5 μM, 5Aza 5 μM on Day 1. Cell counts by automated counter. Mean±SD. D) The anti-proliferative action of Dec and 5Aza was not via early apoptosis. Cell membrane Annexin-V staining measured by flow-cytometry on Day 2. Positive control used etoposide at 400 μg/ml. E) Dec and 5Aza treatment, but not cytarabine, induced morphologic changes of differentiation (decreased nuclear cytoplasmic ratio, increased cytoplasmic complexity). Giemsa-stained cytospins Day 5 (drug added Day 1). F) Dec and 5Aza, but not cytarabine, decreased Myc and increased p27/Cdkn1B. Western blot Day 5 (drug added Day 1). Graph shows results of densometric analysis.

FIG. 4: The addition of THU increased lung cancer cytoreduction and immune-priming over Dec or 5Aza alone. A) Experiment schema. C57/BL6 mice were inoculated via tail vein with LL3 lung cancer cells (0.35×10⁶ cells/mouse. Mice were randomized to PBS, Dec (0.2 mg/kg sc 3×/wk), THU-Dec (10/0.1 mg/kg sc 3×/wk), 5Aza (2 mg/kg sc 3×/wk), or THU-5Aza (10/1 mg/kg sc 3×/wk)(n=5/group). B) THU addition significantly increased time-to-distress over Dec or 5Aza alone. p-values Log Rank test. C) THU addition produced more DNMT1-depletion, MYC reduction and p27 upregulation over Dec or 5Aza alone. Western blots of pooled tumor tissue harvested at euthanasia. D) THU addition decreased nuclear:cytoplasmic ratio over Dec or 5Aza alone. H&E staining of tumor sections obtained at euthanasia. E) THU addition increased tumor infiltration by immune-effector cells over Dec or 5Aza alone. Measured by flow cytometry of homogenized tumor tissue. F) THU addition increased cancer testes expression over Dec or 5Aza alone. Measured by QRT-PCR. G) THU addition increased MHC class 2 expression over Dec or 5Aza alone. H-2K^{b}/H-2D^{b} expression in tumor tissue measured by flow cytometry of dissociated tumor tissue. Bar and whiskers=median IQR. *p<0.05; **p<0.01; ***p<0.001; ****p<0.0001; ns: p>0.05. 2-sided Mann-Whitney test.

FIG. 5: Alternating THU-Dec with THU-5Aza enhanced pharmacodynamic effect and cytoreduction over THU-Dec or THU-5Aza alone. A) Experiment schema. C57BL/6 mice were inoculated via tail vein with LL3-luc lung cancer cells (0.35×10⁶ cells/mouse. After documentation of lung invasion by live-imaging, mice were distributed to PBS, THU-Dec (10/0.1 mg/kg sc 3×/wk), THU-5Aza (10/1 mg/kg sc 3×/wk), and THU-Dec/THU-5Aza (THU-Dec 10/0.1 mg/kg sc 2×/wk then THU-5Aza 10/1 mg/kg sc on the third day). B) Tumor burden before and during treatment with the different regimens. Live-imaging. C) The alternating regimen significantly increased time-to-distress over THU-Dec or THU-5Aza alone. p-values Log Rank test. D) The alternating regimen decreased nuclear:cytoplasmic ratio over THU-Dec or THU-5Aza alone. H&E staining of tumor sections obtained at euthanasia. E) The alternating regimen produced more DNMT1-depletion, Myc reduction and p27 upregulation over THU-Dec or THU-5Aza alone. Western blots of pooled tumor tissue harvested at euthanasia. F) Effects of the different regimens on tumor infiltration by immune-effector cells. Measured by flow cytometry of dissociated tumor tissue. G) Effects of the different regimens on tumor cancer testis antigen expression. Bar and whiskers=median±IQR. ns: p>0.05. 2-sided Mann-Whitney test.

FIG. 6: THU-Dec enhanced the immuno-therapeutic effects of anti-Pd1. C57BL/6 mice were inoculated via tail vein with LL3-luc lung cancer cells (0.35×10⁶ cells/mouse). After documentation of lung invasion by live-imaging, mice (n=5/group) were randomized to treatment with PBS, THU-Dec (10/0.1 mg/kg sc 3×/wk), anti-PD1 (5 mg/kg ip q5d) or anti-PD1+THU-Dec combination. A) Experiment schema. B) Tumor burden before and during treatment with the different regimens. Live-imaging. C) Combination anti-PD1+THU-Dec produced the greatest increases in time-to-distress over the other treatments, including two apparent cures. p-values Log Rank test. D) Cured mice and tumor-naïve control mice were challenged with LL3-luc cells. Tumor engraftment measured by live imaging. E) Effects of the different regimens on tumor infiltration by immune-effector cells. Measured by flow cytometry of dissociated tumor tissue. F) PB T-cell oligoclonality was significantly increased in mice cured by the combination treatment vs vehicle-treated controls. Oligoclonality index: percentage of the overall T-cell population represented by the 1000 most abundant T-cell clones, measured by T-cell receptor (TCR) RNA sequencing. Bar and whiskers=Median IQR; *p<0.05; ns: p>0.05. 2-sided Mann-Whitney test.

FIG. 7: Alternating THU-Dec with THU-5Aza further enhanced the immuno-therapeutic effects of anti-PD1. C57BL/6 mice were inoculated via tail vein with LL3-luc lung cancer cells (0.35×10⁶ cells/mouse). After documentation of lung invasion by live-imaging, mice (n=5/group) were distributed to PBS, THU-Dec 10/0.1 SC 2×/wk followed by THU-5Aza 10/1 mg/kg SC 1×/wk), anti-PD1 (5 mg/kg IP q5d) or anti-PD1+THU-Dec/5Aza combination. A) Experiment schema. B) Tumor burden before and during treatment with the different regimens. Live-imaging. C) Combination anti-PD1+THU-Dec/5Aza produced the greatest increases in time-to-distress over the other treatments, including three apparent cures. p-values Log Rank test. D) Cured mice and tumor-naïve control mice were challenged with LL3-luc cells. Tumor engraftment measured by live imaging. E) Effects of the different regimens on tumor infiltration by immune-effector cells. Measured by flow cytometry of homogenized tumor tissue. F) PB T-cell oligoclonality was significantly increased in mice cured by the combination treatment vs vehicle-treated controls. Oligoclonality index=percentage of the overall T-cell population represented by the 1000 most abundant T-cell clones, measured by T-cell receptor (TCR) RNA sequencing. Bar and whiskers=Median±IQR; *p<0.05; **p<0.01; ns: p>0.05. 2-sided Mann-Whitney test.

FIG. 8. THU-Dec/5Aza alternating treatment and combination epigenetic-immunotherapy were of benefit in the SCLC model, but anti-PD1 alone was of minimal benefit. B6/129 F1 mice were inoculated via tail vein with F1339-luc SCLC cells (0.3×10⁶ cells/mouse). After documentation of tumor invasion by live-imaging, mice (n=5/group) were randomized to PBS, THU-Dec/5Aza, anti-PD1 or anti-PD1+THU-Dec/5Aza combination. A) Experiment schema. B) Tumor burden before and during treatment with the different regimens. Live-imaging. C) Combination anti-PD1+THU-Dec/5Aza produced the greatest increases in time-to-distress over the other treatments; anti-PD1 alone appeared to be of minimal or no benefit. p-values Log Rank test. D) Combination anti-PD1+THU-Dec/5Aza decreased PB granulocytic and monocytic myeloid-derived suppressor cells (G-MDSC and M-MDSC). E) Peripheral blood CD8⁺ and CD4⁺ T-cells were significantly and similarly increased. PB counts of the various cell types, measurements by flow cytometry. Bar and whiskers=Median±IQR. *p<0.05, ns: p>0.05. 2-sided Mann-Whitney test.

FIG. 9. Myeloid malignancies (e.g., acute myeloid leukemia, AML), the only malignancies for which decitabine and 5-azacytidine are currently approved or routinely used as therapy, express up to several-fold higher levels of DCK and UCK2, and have a higher growth fraction (represented by MK167 expression) than non-small cell lung cancer (NSCLC) (adeno=adenocarcinoma; squam=squamous carcinoma). Gene expression by RNA-sequencing by TCGA, AML n=173, NSCLC Adeno n=515, NSCLC squam n=498.

FIG. 10. Effects of Dec or 5Aza vs tetrahydrouridine-Dec (THU-Dec) or THU-5Aza on peripheral blood (PB) immune-suppressor and immune-effector cell numbers after 14 days of treatment. C57BL/6 mice were inoculated via tail vein with LL3 lung cancer cells (0.35×10⁶ cells/mouse). Mice were randomized to PBS, Dec (0.2 mg/kg sc 3×/wk), THU-Dec (10/0.1 mg/kg sc 3×/wk), 5Aza (2 mg/kg sc 3×/wk), or THU-5Aza (10/1 mg/kg sc 3×/wk)(n=5/group). A) PB granulocytic and monocytic myeloid-derived suppressor cells (G-MDSC and M-MDSC) were significantly and similarly decreased (˜4-fold) by the various treatments versus vehicle, while peripheral blood CD8⁺ and CD4⁺ T-cells were significantly and similarly increased. B) In PB, numbers of peripheral blood regulatory T cells (T_{reg}) were not significantly changed by the treatments vs vehicle. C) In tumor tissue, T_{reg} were similarly decreased by all the treatments. D) Suggesting a basis for increased T cell infiltration of tumor, expression of tumor-testes antigens (Mage-A1, Mage-A3, Mage-A6) was increased the most (up to 6-fold) by THU-Dec or THU-5Aza. E) The antigen presenting molecule MHC class I H-2K^{b}/H-2D^{b} was similarly increased by ˜2-fold by all the treatments versus vehicle. Bar and whiskers=median±IQR. *p<0.05; ns: p>0.05. 2-sided Mann-Whitney test.

FIG. 11. THU-Dec/THU-5Aza alternating treatment enhanced the effects over THU-Dec or THU-5Aza alone. C57BL/6 mice were inoculated via tail vein with LL3-luc lung cancer cells (0.35×10⁶ cells/mouse). After documentation of lung invasion by live-imaging, mice (n=5/group) were distributed to PBS, THU/decitabine (10/0.1 mg/kg sc 3×/wk), THU/5-azacytidine (10/1 mg/kg sc 3×/wk), THU/decitabine/5-azacytidine alternating (THU/decitabine 10/0.1 mg/kg sc 2×/wk followed with THU/5-azacytidine (10/1 mg/kg sc on the third day). A) Absolute numbers of peripheral blood G-MDSC and M-MDSC were significantly and similarly decreased for the different treatments versus vehicle, while peripheral blood CD8⁺ and CD4⁺ T-cells were significantly and similarly increased. B) In PB, numbers of peripheral blood regulatory T_{reg} were not significantly changed by the treatments vs vehicle. C) In tumor tissue, T_{reg} were similarly decreased by all the treatments vs vehicle. D) All three treatment regimens similarly increased expression of tumor-testes antigens (Mage-A1, Mage-A3, Mage-A6) vs vehicle. Bar and whiskers=median±IQR. *p<0.05; ns: p>0.05. 2-sided Mann-Whitney test.

FIG. 12. THU-Dec enhanced the immuno-therapeutic effects of anti-Pd1. C57BL/6 mice were inoculated via tail vein with LL3-luc lung cancer cells (0.35×10⁶ cells/mouse. After documentation of lung invasion by live-imaging, mice (n=5/group) were distributed to PBS, THU-Dec (10/0.1 mg/kg SC 3×/wk), anti-Pd1 (5 mg/kg IP q5d) or anti-Pd1+THU-Dec combination. A) Absolute numbers of peripheral blood G-MDSC and M-MDSC were significantly and similarly decreased by the different treatments vs vehicle, while PB CD8⁺ and CD4⁺ T-cells were significantly and similarly increased. B) PB T_{reg} were significantly decreased by anti-Pd1 and the anti-Pd1/THU-decitabine combination vs the other treatments. C) T_{reg} infiltration of tumor was similarly decreased by all treatments vs vehicle but not to a significant extent. D) Dnmt1-depletion, Myc-downregulation and p27/Cdkn1b upregulation was greatest with the combination treatment. Western blot of pooled tumor tissue from multiple mice. Bar and whiskers=median±IQR. *p<0.05; **p<0.01; ****p<0.0001; ns: p>0.05. 2-sided Mann-Whitney test.

FIG. 13. THU-Dec alternating with THU-5Aza further enhanced the immuno-therapeutic effects of anti-Pd1. C57BL/6 mice were inoculated via tail vein with LL3-luc lung cancer cells (0.35×10⁶ cells/mouse). After documentation of lung invasion by live-imaging, mice (n=5/group) were distributed to PBS, THU-Dec (10/0.1 mg/kg SC 2×/wk) followed by THU-5Aza SC 1×/wk, anti-Pd1 (5 mg/kg IP q5d) or anti-Pd1+THU-Dec/5Aza combination. A) Absolute numbers of peripheral blood G-MDSC and M-MDSC were significantly and similarly decreased by the different treatments vs vehicle. B) PB CD8⁺ and CD4⁺ T-cells were significantly and similarly increased by the different treatments vs vehicle. C) PB T_{reg} were non-significantly increased by the different treatments. D) T_{reg} infiltration of tumor was significantly and similarly decreased by all treatments vs vehicle. Bar and whiskers=median±IQR. ns: p>0.05. 2-sided Mann-Whitney test.

FIG. 14: Alternating THU-Dec with THU-5Aza further enhanced the immuno-therapeutic effects of anti-Pd1. C57BL/6 mice were inoculated via tail vein with LL3-luc lung cancer cells (0.35×10⁶ cells/mouse). After documentation of lung invasion by live-imaging, mice (n=5/group) were distributed to PBS, THU-Dec/5Aza, anti-Pd1 or anti-Pd1+THU-Dec/5Aza combination. A) Experiment schema. B) Tumor burden before and after 14 days of treatment with the different regimens. Live-imaging. C) Tumor burden as measured by lung weights was lowest with the combination treatment, although not significantly so vs the other treatments. D) Effects of the different regimens on tumor infiltration by immune-effector cells. Measured by flow cytometry of homogenized tumor tissue. E) T-cell oligoclonality was increased the most by the combination treatment. Oligoclonality index=percentage of the overall T-cell population represented by the 1000 most abundant T-cell clones, measured by T-cell receptor (TCR) diversity analysis of TILs by RNA sequencing. F) Pd1/PD-L1 immune checkpoint gene expression in tumor tissue was increased the most by the combination treatment. Bar and whiskers=median±IQR. *p<0.05; **p<0.01; ***p<0.001; ns: p>0.05. 2-sided Mann-Whitney test.

FIG. 15. Alternating THU-Dec with THU-5Aza further enhanced the immuno-therapeutic effects of anti-Pd1. C57BL/6 mice were inoculated via tail vein with LL3-luc lung cancer cells (0.35×10⁶ cells/mouse). After documentation of lung invasion by live-imaging, mice (n=5/group) were distributed to PBS, THU-Dec/5Aza, anti-Pd1 or anti-Pd1+THU-Dec/5Aza combination. A) Combination epigenetic-immunotherapy produced more DNMT1-depletion, Myc reduction and p27 upregulation over THU-Dec/5Aza or ICB alone. Western blots of pooled tumor tissue harvested at euthanasia. B) Absolute numbers of peripheral blood G-MDSC and M-MDSC were significantly and similarly decreased by the different treatments vs vehicle, while PB immune-effector cells were most significantly increased by combination epigenetic-immunotherapy. C) PB T_{reg} numbers were not significantly altered by the treatments vs vehicle. D) T_{reg} infiltration of tumor was most decreased by combination treatment, but not to a significant extent vs the other treatments. Bar and whiskers=median±IQR. *p<0.05; **p<0.01; ns: p>0.05. 2-sided Mann-Whitney test.

FIG. 16. Effects of the epigenetic and anti-Pd1 treatment on immune checkpoint expression in tumor tissue and TILs. C57BL/6 mice were inoculated via tail vein with LL3-luc lung cancer cells (0.35×10⁶ cells/mouse). After documentation of lung invasion by live-imaging, mice (n=5/group) were distributed to PBS, THU-Dec/5Aza, anti-Pd1 or anti-Pd1+THU-Dec/5Aza combination. Lag3, Ctla4, and Tim3 expression were measured in mRNA extracted from TILs (magnetically selected for CD8 expression), the other checkpoints were measured in mRNA isolated from whole tumor tissue. Bar and whiskers=median±IQR. *p<0.05; ***p<0.001; ns: p>0.05. 2-sided Mann-Whitney test.

FIG. 17: THU-Dec/5Aza epigenetic treatment was beneficial to the mice with SCLC, but ICB with anti-PD1 did not add benefit. B6/129 F1 mice were inoculated via tail vein with F1339-luc lung cancer cells (0.3×10⁶ cells/mouse). After documentation of lung invasion by live-imaging, mice (n=5/group) were distributed to PBS, THU-Dec/5Aza, anti-PD1 or combination anti-PD1+THU-Dec/5Aza. A) Experiment schema. B) Tumor burden before and after 14 days of treatment. Live-imaging. C) Tumor burden after 14 days of treatment measured by liver weights. 2-sided Mann-Whitney test. D) Tumor histological sections after 14 days of treatment. H&E staining, showing decreased nuclear-cytoplasmic ratio and increased necrosis with treatment. E) THU-Dec/5Aza or combination epigenetic-immunotherapy, but not anti-PD1 alone, increased H-2K^{b}/H-2D^{b} expression by tumor tissue. Measured by flow cytometry. F) The different treatments favored oligoclonality of TILs, with the greatest clonal restriction with combination epigenetic-immunotherapy. Oligoclonality index=percentage of the CD8+ T-cell population represented by the 1000 most abundant T-cell clones measured by TCR RNA-sequencing. Bar and whiskers=Median±IQR. *p<0.05; ns: p>0.05. 2-sided Mann-Whitney test.

FIG. 18. THU-Dec/5Aza alternating epigenetic treatment, ICB and combination epigenetic-ICB similarly decreased PB granulocytic and monocytic myeloid-derived suppressor cells (G-MDSC and M-MDSC), while peripheral blood CD8⁺ and CD4⁺ T-cells were significantly and similarly increased. B6/129 F1 mice were inoculated via tail vein with F1339-luc lung cancer cells (0.3×10⁶ cells/mouse). After documentation of tumor invasion by live-imaging, mice (n=5/group) were randomized to PBS, THU-Dec/5Aza, anti-Pd1 or combination anti-Pd1+THU-Dec/5Aza. Bar and whiskers=Median±IQR. **p<0.01; ***p<0.001; ns: p>0.05. 2-sided Mann-Whitney test.

FIG. 19. Several immune checkpoints other than Pd1/PD-L1 were upregulated in SCLC treated with anti-Pd1. B6/129 F1 mice were inoculated via tail vein with F1339-luc lung cancer cells (0.3×10⁶ cells/mouse). After documentation of tumor invasion by live-imaging, mice (n=5/group) were randomized to PBS, THU-Dec/5Aza, anti-Pd1 or combination anti-Pd1+THU-Dec/5Aza. A) Immune-checkpoint expression in SCLC tumor tissue after 14 days of treatment. QRT-PCR. Tumor tissue from 3 individual mice per treatment group. B) Immune-checkpoint expression in TILs. TILs from 4 individual mice per group (magnetically selected for CD8 expression). Gene expression by QRT-PCR. Bar and whiskers=median±IQR. **p<0.01; ***p<0.001; ns: p>0.05. 2-sided Mann-Whitney test.

FIG. 20 describes a first exemplary treatment of staggered decitabine and 5-azacytidine weekly treatment protocol for a human patient with cancer over a treatment period of multiple weeks. This protocol includes the possibility of skipping treatment a given week if absolute neutrophil count (ANC) is too low. In such instances, the patient can be administered G-CSF.

The exemplary regimen includes the following. Oral THU at about 10 mg/kg administered 60-300 minutes before oral decitabine at about 0.16 mg/kg on day (e.g., Thu at breakfast, decitabine at lunch). Then, oral THU at about 10 mg/kg administered 60-300 minutes before oral 5-azazytidine at about 1.6 mg/kg on Day 4 (e.g., THU at breakfast, 5-azacytidine at lunch). If the AN is less than 0.5×10⁹/L, treatment is held for that week and G-CSF 200-480 ug is administered instead. If neutropenia is recurrent, consider routinely reducing drug treatment to 3 of 4 weeks each month, with G-CSF administration routinely in the week.

FIG. 21 describes a second exemplary treatment staggered decitabine and 5-azacytidine weekly treatment protocol for a human patient with cancer over a treatment period of multiple weeks. In this protocol, there is an induction period where the patient is administered THU and then decitabine 60 minutes later for five consecutive days, and then G-CSF (NEULASTA neutropenia prophylaxis) anytime between days 5-9. The exemplary regimen includes the following. In week 1: i) oral Thu at about 10 mg/kg is administered 60 minutes before oral decitabine at about 0.2 mg/kg for 5 consecutive days, and ii) Neulasta neutropenia prophylaxis is recommended, to be administered anytime between Days 5-9 of the cycle. Week 2: if there are no greater than or equal to grade 3 treatment-related non-hematologic toxicities, oral Thu-decitabine is administered at the same dose in week 2 according to the following schema: i) baseline ANC in week 2 is 0.5-1.0×10⁹/L, administer for 2 consecutive days; ii) baseline ANC in week 2 is greater than 1.0-1.5×10⁹/L, administer for 3 consecutive days; iii) baseline ANC in week 2 is greater than 1.5-2.0×10⁹/L, administer for 4 consecutive days; and iv) baseline ANC in week 2 is greater than 2.0×10⁹/L, administer for 5 consecutive days. Weeks 3 and 4, if there are no greater than or equal to grade 3 treatment-related non-hematologic toxicities and the ANC is greater than 0.5×10⁹/L, administer oral Thu-decitabine at the same dose for 2 consecutive days. Induction cycles can be repeated if clinically indicated (e.g., in a patient with a partial response but who is still symptomatic and in whom the first induction cycle was well tolerated; or in a patient with disease previously controlled by induction then Maintenance but subsequently relapsing while on maintenance. Cycle 2 onward—maintenance cycles: Oral Thu about 10 mg/kg is administered 60 minutes before oral decitabine at about 0.2 mg/kg two times per week on consecutive days if there are no greater than or equal to grade 3 treatment-related non-hematologic toxicities and the ANC is greater than 0.5×10⁹/L (if the ANC is less than 0.5×10⁹/L, treatment is held until recovery above this threshold then resumed with a dose reduction.

FIG. 22 describes a third exemplary treatment staggered decitabine and 5-azacytidine weekly treatment protocol for a human patient with cancer over a treatment period of multiple weeks. This protocol has a first induction protocol and potential second induction protocol as described in FIG. 22. The exemplary regimen includes the following. In week 1: i) oral Thu at about 10 mg/kg is administered 60 minutes before oral decitabine at about 0.2 mg/kg for 5 consecutive days, and ii) Neulasta neutropenia prophylaxis is recommended, to be administered anytime between Day 5 or 6 of the cycle. Week 2: if there are no greater than or equal to grade 3 treatment-related non-hematologic toxicities, oral Thu-decitabine is administered at the same dose in week 2 according to the following schema: i) baseline ANC in week 2 is 0.5-1.0×10⁹/L, administer for 2 consecutive days; ii) baseline ANC in week 2 is greater than 1.0-1.5×10⁹/L, administer for 3 consecutive days; iii) baseline ANC in week 2 is greater than 1.5-2.0×10⁹/L, administer for 4 consecutive days; and iv) baseline ANC in week 2 is greater than 2.0×10⁹/L, administer for 5 consecutive days. Weeks 3 and 4, if there are no greater than or equal to grade 3 treatment-related non-hematologic toxicities and the ANC is greater than 0.5×10⁹/L, administer oral Thu-decitabine at the same dose for 2 consecutive days. Cycle 2—potentially a second induction cycle versus a maintenance cycle. Repeat induction as per the schema above if CA19-9 is not decreased by at least 10% from baseline or if clinically indicated, otherwise commence maintenance. Induction cycles can be repeated even after cycle 2 if the treating clinical team judges such a repeat to be in the best interest of the patient (e.g., for previously controlled disease relapsing during maintenance). Cycles 2 and 3 onward—maintenance cycles: Oral Thu about 10 mg/kg is administered 60 minutes before oral decitabine at about 0.2 mg/kg two times per week on consecutive days if there are no greater than or equal to grade 3 treatment-related non-hematologic toxicities and the ANC is greater than 0.5×10⁹/L (treatment is held until recovery of ANC to greater than 0.5×10⁹/L).

FIG. 23 shows DNMT1 is not depleted at clinical relapse or with in vitro resistance. A) Key pyrimidine metabolism enzymes shown to be critical to DNMT1-depleting activity of decitabine (Dec) or 5-azacytidine (5Aza) in vitro. dCDP=deoxycytidine diphosphate; CDP=cytidine diphosphate; dCTP—deoxycytidine triphosphate. B) Decitabine (Dec) or 5-azacytidine (5Aza) decreased bone marrow DNMT1 at response (green) vs pre-treatment (dark blue) but not at relapse (red). Serial bone marrow biopsies from the same patient were cut onto the same slide, stained for DNMT1, and the number of DNMT1-positive nuclei was quantified objectively using ImageIQ software in 13 patients and positive/negative controls (tissue blocks of HCT116 wild-type and DNMT1-knockout cells respectively). D=days of therapy. Pre-Rx=pre-treatment; HI=hematologic improvement; CR=complete remission; SD=stable disease; Rel.=relapse. Mean±SD of ×3 image segments (cellular regions) per sample; p-value paired t-test, 2-sided. C) Expression of key pyrimidine metabolism enzymes at relapse/progression on Dec. Schema shows key enzymes that favor (green) or impede (red) Dec or 5Aza conversion into the DNMT1-depleting Aza-dCTP. Bone marrow cells aspirated pre-treatment and at relapse/progression on Dec (13 patients, median duration of therapy 175 days, range 97-922) or 5Aza (14 patients, median duration of therapy 433 days, range 61-1155) were analyzed by QRT-PCR. Mean±SD technical replicates×3. Paired t-test, 2-sided. D) Expression by Western blot of DNMT1 and key pyrimidine metabolism enzymes in Dec-resistant AML cells (THP1, K562, OCI-AML3 and MOLM13), and in parental THP1 AML cells treated with vehicle, Dec or 5-azacytidine (5Aza). Western blots.

FIG. 24 shows Dec and 5Aza cause nucleotide imbalances and metabolic compensations. A) Experiment schema. Vehicle, natural deoxycytidine (dC) 0.5 uM, natural cytidine (C) 5 uM, Dec 0.5 uM, or 5Aza 5 uM were added once to AML cells at 0 hours. B) Cell counts. By automated counter. Means±SD for 3 independent biological replicates for each cell line. C) Dec and 5Aza have opposite effects on dCTP levels. Measured by LCMS/MS 24 hrs after addition of Dec or 5Aza. Analyses of 2 or more independent nucleotide extractions from 3 different AML cells lines. Means±SD; p-values paired t-test, 1-sided. D) Gene expression 72 hours after Dec or 5Aza. Gene expression by QRT-PCR, relative to average expression in vehicle-treated controls. Means±SD for 3 independent biological replicates in each of 3 AML cell lines; p-values unpaired t-test vs vehicle, 2-sided. E) Western blot 72 hours after Dec or 5Aza. AML cells THP1, OCI-AML3 and K562. Western blots were reproduced in three independent biological replicates.

FIG. 25 shows DCK is important for maintaining dCTP and UCK2 for maintaining dTTP levels. A) DCK and UCK2 knockout (KO) were confirmed by Western blot. HAP1 leukemia cells, KO by CRISPR-Cas9. B) DCK-KO lowers dCTP and UCK2-KO lowers dTTP. Analysis of independent nucleotide extractions. Means±SD; p-values unpaired t-test, 2-sided. C) Sensitivity of Wildtype, DCK-KO and UCK2-KO HAP1 leukemia cells to Dec vs 5Aza. Means±SD of 3 independent biological replicates.

FIG. 26 shows Impact on efficacy of adding CDA and/or de novo pyrimidine synthesis inhibitors. NSG mice were tail-vein inoculated with patient-derived AML cells (1×106 cells/mouse) and randomized to (i) PBS vehicle control; (ii) CDA-inhibition by intra-peritoneal (IP) tetrahydrouridine (THU) and de novo pyrimidine synthesis inhibition by IP thymidine (dT); (iii) Dec; (iv) THU and Dec; (v) THU, Dec and dT (n=5/group). PBS, THU+dT mice were euthanized for distress on D42, and other mice were sacrificed for analysis on D63. A) Experiment schema. B) Femoral bones (from 2 of 5 mice/group). White=leukemia replacement, reddish=functional hematopoiesis. C) Bone marrow human (hCD45) and murine (mCd45) myelopoiesis content. Flow-cytometry. Median±IQR. p-value Mann-Whitney test 2-sided. D) Blood counts before treatment and at euthanasia/sacrifice. Measured by Hemavet. Median±IQR. E) Spleen AML burden as measured by spleen weights at euthanasia/sacrifice. Median±IQR. p-value Mann-Whitney test 2-sided. F) Spleen histology. Hematoxylin-Eosin stain of paraffin-embedded sections. Magnification 400×. Leica DMR microscope.

FIG. 27 shows Comparison of THU/Dec alone vs THU/5Aza alone vs THU/Dec alternating with THU/5Aza in 4 week cycles. NSG mice were tail-vein inoculated with patient-derived AML cells (1×106 cells/mouse) and on Day 9 after inoculation randomized to the treatments as shown (n=7/group). Mice were euthanized if there were signs of distress. A) Experiment schema; B) Time-to-distress and euthanasia. C) Bone marrow human (hCD45) and murine (mCd45) myelopoiesis content. Femoral bones flushed after euthanasia. Measured by flow-cytometry. Median±IQR. D) Spleen weights at time-of-distress/euthanasia. Median±IQR. E) Spleens at the time-of-distress/euthanasia.

FIG. 28 shows Alternating THU/Dec with THU/5Aza week to week. NSG mice were tail-vein inoculated with patient-derived AML cells (1×106 cells/mouse) and randomized to the treatments shown (n=5/group). Blood counts were obtained periodically by tail-vein phlebotomy. Mice were euthanized for signs of distress. A) Experiment schema; B) Time-to-distress. Log-rank test. C) Serial blood counts. Measured by Hemavet. Median±IQR. D) Bone marrow replacement by AML. Bone marrow human and murine CD45+ cells measured by flow-cytometry (FIG. 32) after euthanasia (time-points panel B). Median±IQR. p-value Mann-Whitney test 2-sided. E) DNMT1 was not depleted from AML cells at progression (time-points panel B) but was depleted at time-of-response (bone marrow harvested at Day 63 in a separate experiment). Flow cytometry (FIG. 33). F) Pyrimidine metabolism gene expression in bone marrow AML cells. QRT-PCR using human gene specific primers, bone marrow harvested after euthanasia. p-values vs vehicle, unpaired t-test, 2-sided.

FIG. 29 shows time to emergence of AML cells exponentially proliferating in presence of clinically relevant concentrations of decitabine (Dec). Cell counts by automated counter.

FIG. 30 shows a time-course analysis suggested that peak changes in pyrimidine metabolism enzyme protein levels occurred between 48-96 hours after a single exposure to decitabine (Dec) 0.25 μM or 5-azacytidine (5Aza) 2.5 uM. A) Western blots for DNMT1, UCK2, DCK, CDA, CAD, TYMS and GAPDH before and up to 96 hours after addition of decitabine (Dec) 0.25 μM or B) 5-azacytidine 2.5 uM to THP1 and OCI-AML3 cells (added once at 0 hours). C) Off-target pathways of Dec and 5Aza. D) Effects of Dec and 5Aza on RRM2A, RRM2B and CTPS1 protein levels. Western blots 72 hours after addition of a single dose of Dec 0.25 μM or 5Aza 2.5 uM. Western blots were reproduced in biological replicates.

FIG. 31 shows Impact of decitabine scheduling to avoid vs coincide with DCK troughs. NSG mice were tail-vein inoculated with patient-derived AML cells (1×10⁶ cells/mouse) and randomized on day 9 after inoculation to (i) PBS vehicle control; (ii) subcutaneous (SC) decitabine (Dec) 0.3 mg/kg (mpk) on Day 1 and 2 of each week (D1,2); (iii) Dec 0.3 mpk on Day 1 and 4 of each week (D1,4)(n=7/group). Mice were euthanized/sacrificed on day 45 when PBS treated mice showed signs of distress. A) Experiment schema; B) Bone marrow cell cytospin and Giemsa-stain at Day 45. Normal=normal NSG mouse bone marrow; Leica DMR microscope, 630×. C) Percentage of human CD45+(huCD45) positive cells in bone marrow. Flow cytometry. Median±IQR. p-value Mann-Whitney test 2-sided. 5 mice in each treatment group analyzed. D) Spleens at Day 45. Normal=spleen from normal NSG mouse. E) Spleen histology. Hematoxylin-Eosin stain of paraffin-embedded sections. Leica DMR microscope, 400×. F) Spleen weights. Median±IQR. p-value Mann-Whitney test 2-sided.

FIG. 32 shows impact of decitabine scheduling to avoid or coincide with DCK troughs. NSG mice were tail-vein inoculated with patient-derived AML cells (1×10⁶ cells/mouse) and on day 9 after inoculation randomized to (i) PBS vehicle control; (ii) subcutaneous (SC) decitabine (Dec) 0.2 mg/kg (mpk) on Day 1 and 2 of each week (D1,2); (iii) Dec 0.2 mpk on Day 1 and 4 of each week (D1,4)(n=4/group). Mice were euthanized for signs of distress. A) Experiment schema; B) Time-to-distress. p-value Log-rank test. C) Bone marrow human leukemia cell burden at time-of-distress. Flow cytometry for human CD45+ cells. Median±IQR. p-value Mann-Whitney test 2-sided. D) Spleens at time-of-distress. E) Spleen weights at time-of-distress. Median±IQR. p-value Mann-Whitney test 2-sided.

FIG. 33 shows The addition of hydroxyurea to inhibit ribonucleotide reductase did not augment THU-decitabine activity; Distributed administration of THU-decitabine 2×/week was superior to pulse-cycled administration for 5 consecutive days every 4 weeks. NSG mice were tail-vein inoculated with patient-derived AML cells (1×10⁶ cells/mouse) and on Day 5 after inoculation randomized to the treatments as shown (n=7/group). Mice were euthanized if there were signs of distress. A) Experiment schema to evaluate potential benefit of adding hydroxyurea to inhibit ribonucleotide reductase; B) Bone marrow human (hCD45) and murine (mCd45) myelopoiesis content. Femoral bones flushed after termination of the experiment at the time PBS-treated mice developed signs of distress. Measured by flow-cytometry. Median±IQR. P-value 2-sided Mann-Whitney test. n.s.=not significant. C) Experiment schema to compare metronomic administration 2×/week versus pulse-cycled administration for 5 consecutive days every 4 weeks; D) Bone marrow human (hCD45) and murine (mCd45) myelopoiesis content. Femoral bones flushed after termination of the experiment at the time PBS-treated mice developed signs of distress. Measured by flow-cytometry. Median±IQR. P-value 2-sided Mann-Whitney test. n.s.=not significant.

FIG. 34 shows THU/decitabine vs THU/5-azacytidine vs THU/decitabine/5-azacytidine. NSG mice were tail-vein inoculated with patient-derived AML cells (1×10⁶ cells/mouse) and on Day 9 after inoculation randomized to the treatments as shown (n=5/group). All mice were euthanized or sacrificed when the vehicle-treated group became distressed at Day 45. A) Experiment schema; B) Giemsa stained cytospins of bone marrow cells. Flushed from femoral bones after euthanasia. Magnification 630×. Leica DMR microscope. C) Bone marrow human (hCD45) and murine (mCd45) myelopoiesis content. Measured by flow-cytometry. Median±IQR. D) Spleen weights at time-of-distress/euthanasia. Median±IQR. E) Spleens.

### DETAILED DESCRIPTION

Provided herein are compositions, systems, kits, and methods for treating a patient with cancer by alternate administration of decitabine and 5-azacytidine, or administration of decitabine two times per week on consecutive days, which is generally timed to bypass auto-dampening and exploit cross-priming. Such administration is combined with an inhibitor of the enzyme cytidine deaminase (e.g., tetrahydrouridine) that otherwise rapidly catabolizes decitabine and 5-azacytidine. In certain embodiments, the time between cycles of decitabine and 5-azacytidine administration is about three to four days or five to ten days.

The benefits of immune checkpoint blockade (ICB) to treat lung cancer are limited to a minority of patients, with some cancers demonstrating paradoxical hyper-growth. There is thus a need for complementary treatments that cytoreduce cancers but spare immune-effectors. Inhibition of the epigenetic regulator DNA methyltransferase 1 (DNMT1) has been validated to do this while also increasing cancer immune-visibility. Moreover, the clinical pro-drugs decitabine and 5-azacytidine, after processing by pyrimidine metabolism into a nucleotide analog, can deplete DNMT1. Unfortunately, clinical trials combining these pro-drugs with ICB have produced disappointing results. Metabolic control mechanisms sense and preserve nucleotide balances. Therefore, work conducted during development of embodiments herein examined reactions of the pyrimidine metabolism network in lung cancer cells to decitabine and 5-azacytidine inputs and found that expression of enzymes essential to nucleotide conversion shifted adaptively, automatically dampening DNMT1-depletion. However, responses to the deoxyribonucleoside decitabine primed for activity of the ribonucleoside 5-azacytidine and vice versa. Thus, in murine models of disseminated non-small cell and small cell lung cancer, alternating decitabine with 5-azacytidine, timed to bypass auto-dampening and exploit cross-priming instead, combined with an inhibitor of the enzyme cytidine deaminase that rapidly catabolizes decitabine/5-azacytidine in vivo, significantly increased tumor DNMT1-depletion, cytoreduction, antigen presentation, T-cell infiltration/oligoclonality, and time-to-distress, without off-target cytotoxic effects. Then, combination with ICB produced complete tumor regressions that resisted tumor re-challenge (‘immune-memory’).

There numerous advantages to the systems and methods described herein. Metabolic control mechanisms sense and preserve nucleotide balances. Work conducted during development of embodiments herein examined pyrimidine metabolism reactions to nucleoside inputs and discovered that expression of enzymes essential to nucleoside uptake shifted adaptively to dampen drug uptake. Fortuitously, however, responses to the deoxyribonucleoside decitabine primed for uptake of the ribonucleoside 5-azacytidine and vice versa. Thus, in murine models of disseminated non-small cell and small cell lung cancer, alternating decitabine with 5-azacytidine, timed to bypass auto-dampening and exploit cross-priming, combined with an inhibitor of the enzyme cytidine deaminase that otherwise rapidly catabolizes decitabine/5-azacytidine, significantly increased tumor DNMT1-depletion, antigen presentation, T-cell infiltration/oligoclonality, cytoreduction and time-to-distress. Specifically, decitabine alone was ineffective in a pre-clinical in vivo model of cancer localized to the liver, but co-administration of lower doses of decitabine with an inhibitor of CDA, tetrahydrouridine (THU), eliminated cancer cell sanctuary in CDA-rich liver, importantly, without bone marrow cytotoxicity. Alternating decitabine with 5-azacytidine and combination of both with an inhibitor of CDA, significantly enhanced DNMT1-depletion from NSCLC and SCLC cells in vivo. Regimens optimized in this way preserved a non-cytotoxic mechanism-of-action that spared immune-effectors and synergized with ICB, to produce complete tumor regressions that resisted tumor re-challenge. The optimized DNMT1-depleting regimens produced greater upregulation of antigen presenting MHC class I molecules (MHC I H-2 Kb/H-2 db) and also cancer-testis antigens (Magea1-a4) (that are displayed in MHC class I), and infiltration of tumor by cytotoxic T-cells.

### EXAMPLES

### Example 1

### Combination Epigenetic-Immunotherapy Generates Anti-Cancer Immune Memory in Murine Models of Lung Cancer

This Example describes time delayed dosing of decitabine and 5-azacytidine for the treatment of lung cancer in a mouse model.

### Results

Pyrimidine Metabolism Enzymes that Determine Decitabine and 5-Azacytidine Sensitivity

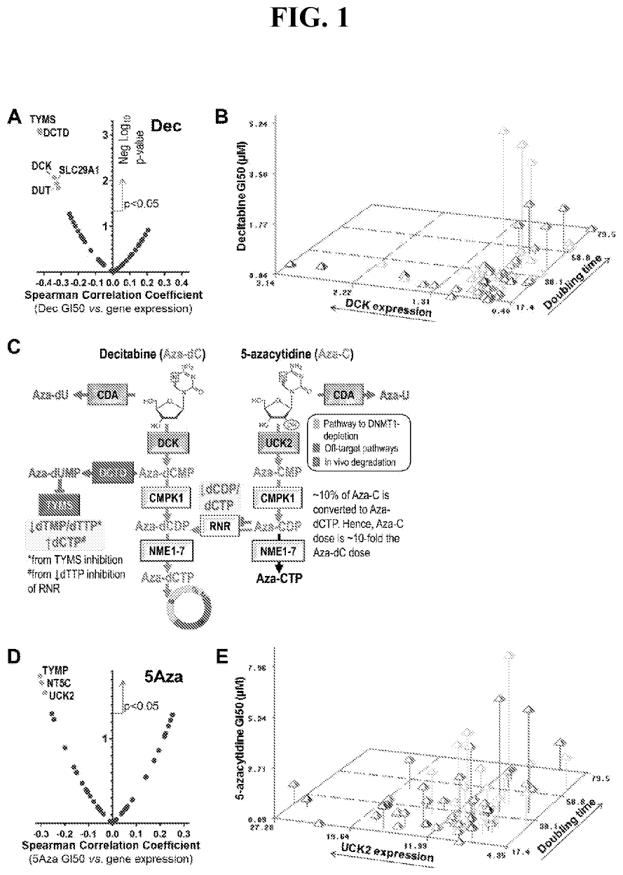

To identify pyrimidine metabolism enzymes most relevant to the activity of decitabine and 5-azacytidine in cells, the expression of 45 known pyrimidine metabolism enzymes and transporters were examined for correlations with decitabine or 5-azacytidine sensitivity (concentrations of decitabine or 5-azacytidine needed to produce 50% growth inhibition [GI50] of cancer cell lines in the NC160 panel). Growth inhibition by decitabine (lower GI50) correlated most strongly with expression of thymidylate synthase (TYMS) (Spearman correlation coefficient [r]=−0.42, p=0.0008), deoxycytidine deaminase (DCTD) (r=−0.42, p=0.0009) and DCK (r=−0.34, p=0.009) (FIG. 1A). DCK phosphorylates decitabine into a deoxycytidine monophosphate (dCMP) analog, Aza-dCMP—in the ‘on-target’ DNMT1-depleting pathway, Aza-dCMP is phosphorylated twice more into Aza-dCTP that depletes DNMT1 (FIG. 1C) (14). In an ‘off-target’ pathway, DCTD deaminates some of this Aza-dCMP into a deoxyuridine monophosphate (dUMP) analog, Aza-dUMP, that potently inhibits TYMS (17-19), an action that decreases deoxycytidine triphosphate (dTTP) (TYMS supplies cells with dTTP). The decrease in dTTP in turn has been shown to increase deoxycytidine triphosphate (dCTP) by dis-inhibiting ribonucleotide reductase (17-19) (FIG. 1C).

Growth inhibition by 5-azacytidine most strongly correlated with expression of TYMP (r=−0.34, p=0.009), NT5C (r=−0.3, p=0.022) and UCK2 (r=−0.28, p=0.029) (FIG. 1D, E). (14). UCK2 phosphorylates 5-azacytidine into a cytidine monophosphate (CMP) analog, Aza-CMP; NT5C and TYMP in tandem can convert some of this Aza-CMP into a pyrimidine analog that has been reported to inhibit de novo pyrimidine synthesis, an ‘off-target’ effect that decreases dCTP (20). In the on-target pathway, Aza-CMP is phosphorylated to a cytidine diphosphate (CDP) analog, Aza-CDP, that is reduced by ribonucleotide reductase to Aza-dCDP, that is phosphorylated into the Aza-dCTP that depletes DNMT1 (FIG. 1C). The on-target pathway incorporates an off-target effect of partial inhibition of ribonucleotide reductase (21) (FIG. 1C), which also decreases dCTP (21).

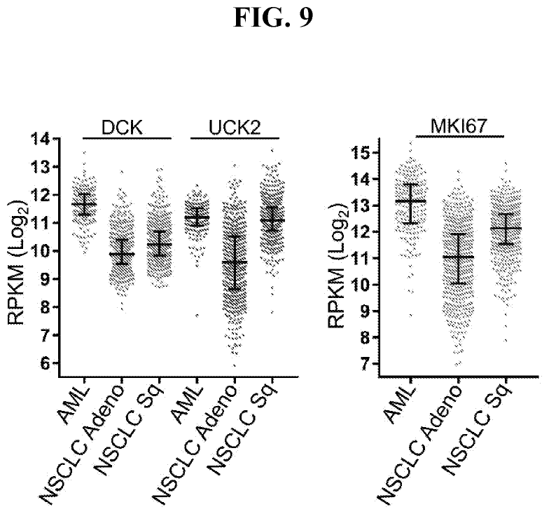

In keeping with a drug efficacy requirement of incorporating into DNA, growth inhibition by both decitabine and 5-azacytidine significantly inversely correlated with cell doubling-time (r=0.30, p=0.021 and r=0.29, p=0.024 respectively) (FIG. 1B, E). Linking these observations to clinical practice, myeloid malignancies that are currently the only approved indication for decitabine or 5-azacytidine, express several-fold higher levels of DCK and UCK2 at baseline, and have a higher growth fraction, than lung cancers (FIG. 9).

Adaptive Responses of the Pyrimidine Metabolism Network to Decitabine or 5-Azacytidine Inputs

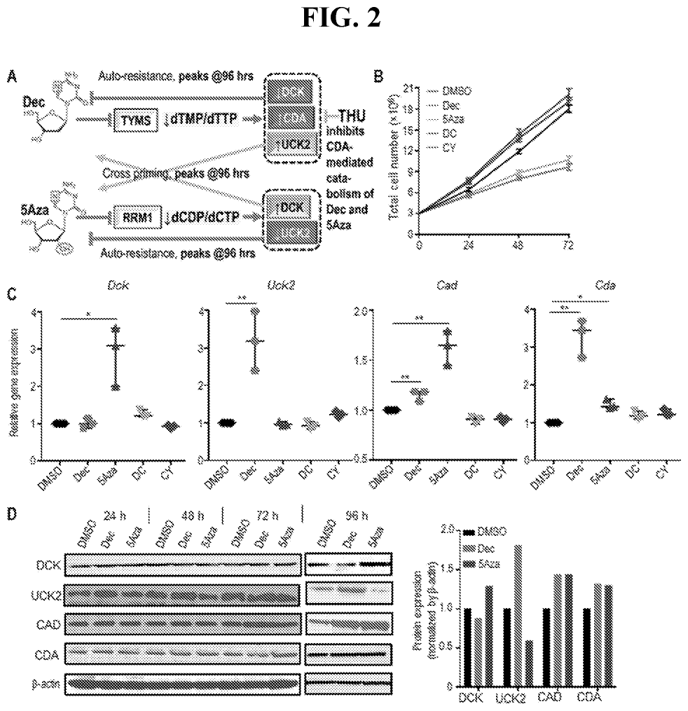

Thus, decitabine and 5-azacytidine, despite a common on-target action of DNMT1-depletion, have separate off-target actions that drive cellular dCTP levels in opposite directions (FIG. 1C, 2A) (16-19, 21). To evaluate if these distinct actions induce specific adaptive responses by the pyrimidine metabolism network that might impact drug metabolism, expression of Dck, Uck2, Cda and Cad were measured using serial QRT-PCR and Western blots in a murine p53-null SCLC cell line (F1339) treated in vitro with DMSO (vehicle), decitabine 0.5 μM, 5-azacytidine 5 μM, natural deoxycytidine 0.5 μM, or natural cytidine 5 μM (F1339 cells were derived from a tumor established by intra-tracheal instillation of Adeno-CMV-Cre in Rb^{lox}/p53^{lox} mice (22)). Decitabine and 5-azacytidine, but not vehicle, natural deoxycytidine or natural cytidine, significantly decreased F1339 proliferation (FIG. 2B). Decitabine and 5-azacytidine, but not vehicle, natural deoxycytidine or cytidine, induced rapid, consistent and distinct changes in expression of these enzymes: decitabine significantly increased Uck2, Cda and Cad expression several-fold, while 5-azacytidine increased Dck, Cda and Cad expression several-fold, with mRNA expression changes peaking at ˜72 hours after drug exposure (FIG. 2C). Protein levels tracked the mRNA changes and peaked at ˜96 hours after drug exposure (FIG. 2D).

p53-Null F1339 Cells are Chemorefractory but Sensitive to Decitabine and 5-Azacytidine

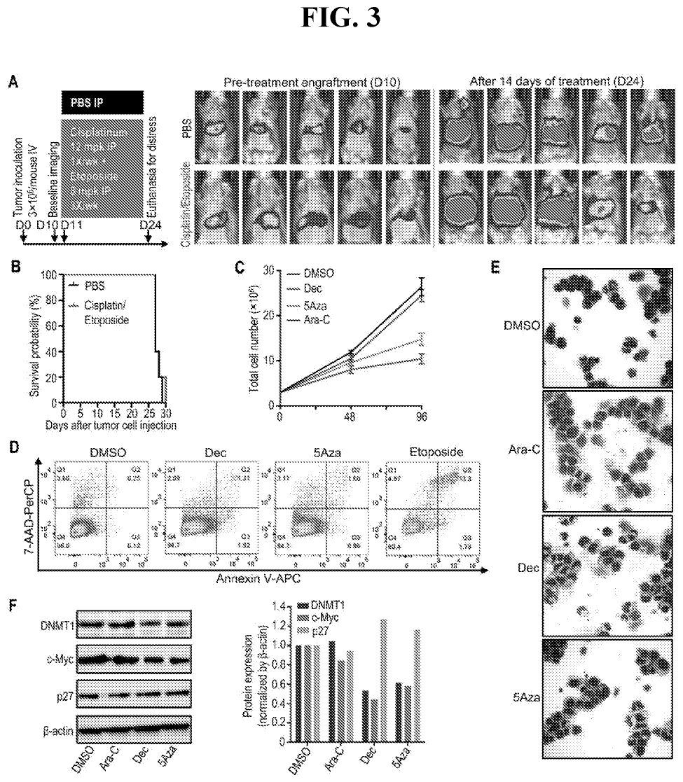

F1339 cells expressing luciferase (F1339-luc) were inoculated via tail vein into B6/129 F1 mice, and after confirmation of tumor engraftment by bioluminescent imaging, the mice commenced treatment with high doses of etoposide and cisplatin, which are used to treat SCLC clinically (FIG. 3A). This aggressive DNA-damaging/apoptosis-based treatment did not benefit the mice—there was no tumor cytoreduction (FIG. 3A) or survival gain to the mice receiving this treatment vs vehicle-treated mice (FIG. 3B). Similarly, in vitro, a clinically relevant concentration of the DNA damaging/apoptosis-inducing deoxycytidine analog cytarabine (Ara-C, 0.5 μM) did not reduce proliferation of the p53-null SCLC cells (FIG. 3C). In contrast, concentrations of the deoxycytidine analog decitabine equimolar to that of cytarabine, and 5-azacytidine at 5 μM (a clinically relevant and safe concentration of this ribonucleotide analog), did significantly impede the SCLC cell proliferation (FIG. 3C). These anti-proliferative actions of decitabine and 5-azacytidine were not by apoptosis-induction, evidenced by unaltered cell membrane Annexin-V staining 24 hours after initiation of drug exposure (FIG. 3D). Instead, decitabine and 5-azacytidine treatment, but not cytarabine, induced morphologic changes in the cells consistent with differentiation induction (decreased nuclear cytoplasmic ratio, increased cytoplasmic complexity) (FIG. 3E). Also consistent with a differentiation-based mechanism-of-action, decitabine and 5-azacytidine, but not cytarabine, decreased Myc protein, the master transcription factor regulator of cell growth and division, and simultaneously increased p27/CDKN1B protein, the canonical cyclin-dependent kinase inhibitor that mediates cell cycle exits by differentiation (FIG. 3F).

### Addition of the CDA-Inhibitor THU to Decitabine or 5-Azacytidine Enhanced Lung Cancer Dnmt1-Depletion, Cytoreduction and Antigen Presentation In Vivo

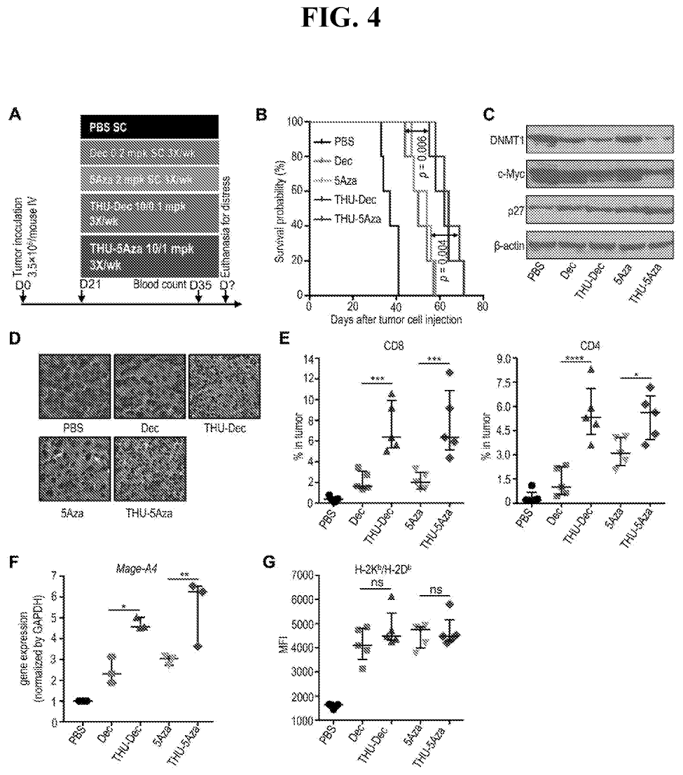

The catabolic enzyme CDA deaminates decitabine and 5-azacytidine into uridine nucleoside analogs that do not deplete DNMT1 but instead cause off-target anti-metabolite effects (FIG. 1C). CDA is highly expressed at baseline in many normal solid organs (e.g., lungs), forming an enzyme barrier impeding activity of decitabine and 5-azacytidine in these tissues in vivo (14). CDA is also rapidly induced by exposure of cancer cells to decitabine (FIG. 2). We therefore evaluated the utility of adding the CDA-inhibitor tetrahydrouridine (THU) to decitabine or 5-azacytidine for in vivo therapy of lung cancer. A murine syngeneic model of disseminated lung cancer was established by tail-vein inoculation of C57/BL6 mice with Lewis lung tumor cells (LL3). At day 21 after tumor inoculation, mice were randomized to treatment with: (i) vehicle; (ii) decitabine; (iii) 5-azacytidine; (iv) THU and decitabine; or (v) THU and 5-azacytidine (FIG. 4A). The drugs were administered by regimens that are not cytotoxic to bone marrow as we have previously described (9, 26, 29). Decitabine or 5-azacytidine increased time-to-distress of the mice by about 20 days over vehicle, and the addition of THU significantly increased this to 30 days for both decitabine and 5-azacytidine (FIG. 4B). Tumor tissues harvested at the time-of-euthanasia were analyzed for achievement of the intended molecular pharmacodynamic effect of Dnmt1-depletion. Dnmt1 protein levels were lowest in the tumor tissues from mice treated with THU-decitabine or THU-5-azacytidine (FIG. 4C). Similarly, the greatest decreases in Myc protein (FIG. 4C), and greatest increases in p27/Cdkn1b (FIG. 4C), and greatest decreases in tumor cell nuclear/cytoplasmic ratio (a morphologic feature of cell differentiation) (FIG. 4D), occurred with the THU combined with decitabine or 5-azacytidine over the respective single agents.

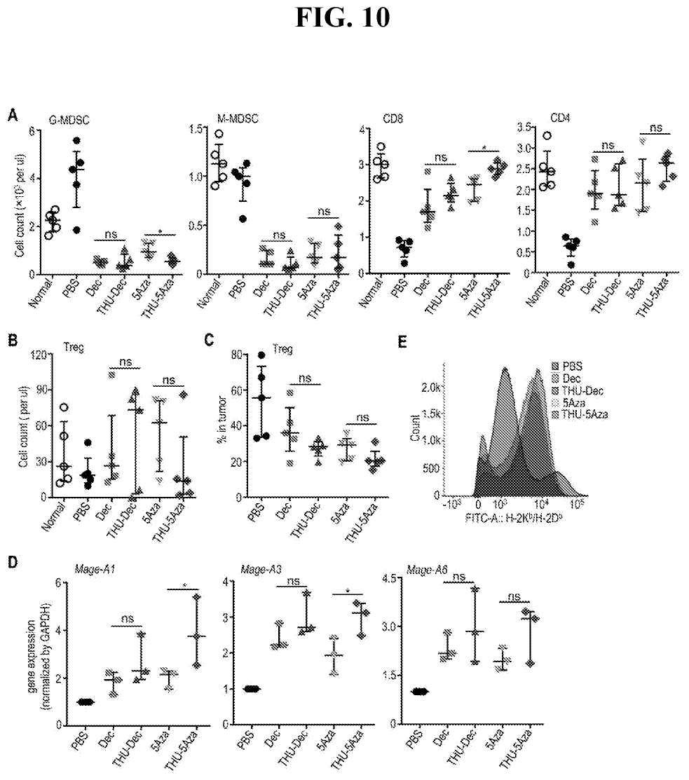

Besides direct effects on tumor tissues, we also examined the effects of these treatments on peripheral blood immune-effector and immune-suppressor cell numbers. Peripheral blood CD8+ and CD4+ T-cells were significantly and similarly increased (FIG. 10A), while peripheral blood granulocytic and monocytic myeloid-derived suppressor cells (G-MDSC and M-MDSC) were significantly and similarly decreased (˜4-fold), by all the treatments vs vehicle (FIG. 10A). In tumor tissues, THU-decitabine and THU-5-azacytidine significantly increased infiltration by CD8+ and CD4+ T-cells relative to decitabine or 5-azacytidine alone (FIG. 4E). Tumor infiltration by T regulatory cells (T_{reg}) was similarly decreased by all the treatments (FIG. 10C), although numbers of peripheral blood regulatory T cells (T_{reg}) did not change significantly (FIG. 10B). Suggesting a possible basis for increased T cell infiltration of tumor, expression levels of cancer-germline antigens (Mage-A1, Mage-A3, Mage-A4, Mage-A6) were increased by all the treatments vs vehicle, but most (up to 6-fold) by THU-decitabine or THU-5-azacytidine (FIG. 4F, 10D). The antigen presenting molecule MHC class I H-2K^{b}/H-2D^{b} was similarly increased ˜2-fold by all treatments (FIG. 4G, TOE).

Alternating Decitabine with 5-Azacytidine Further Enhanced Efficacy

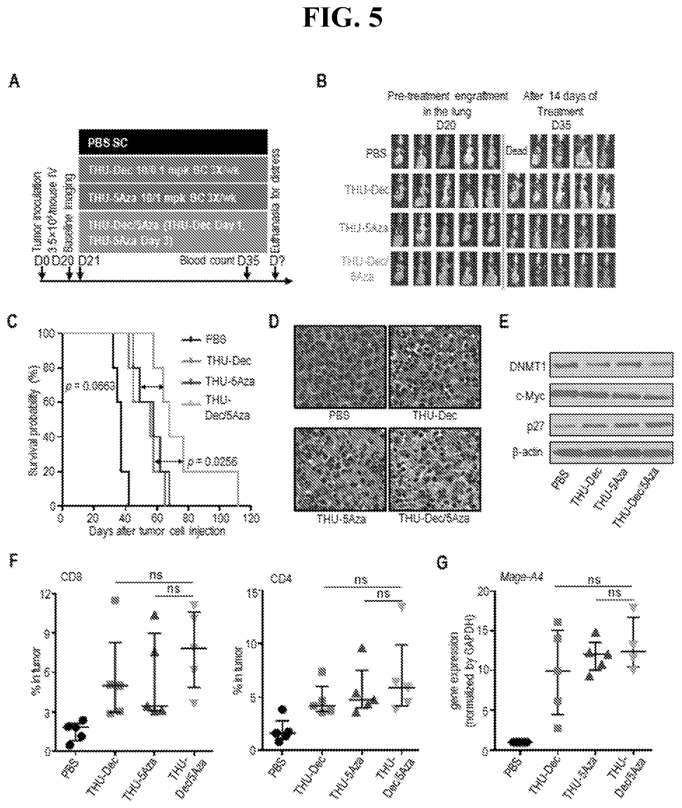

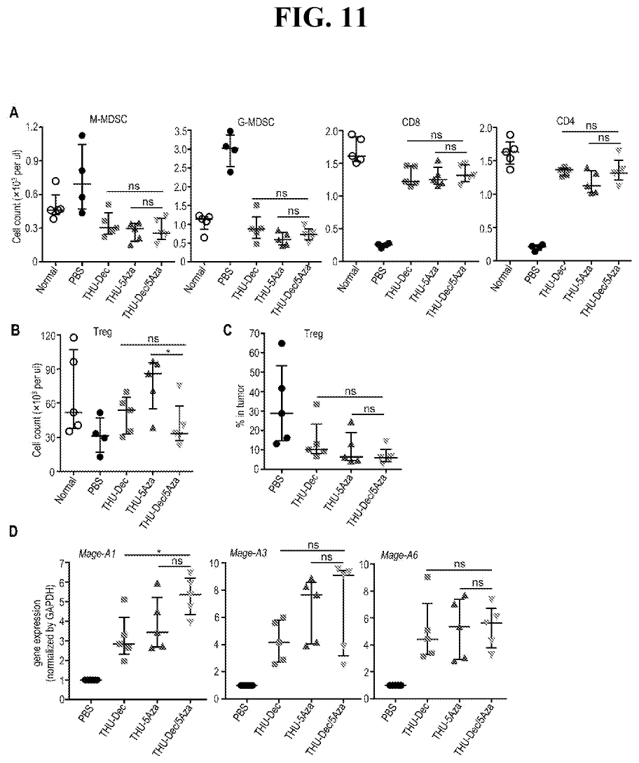

Decitabine and 5-azacytidine caused downregulations of their respective activity-rate-limiting enzymes, DCK and UCK2, but simultaneously upregulated respectively UCK2 and DCK, an effect we refer to as ‘cross-priming’ (FIG. 2). Thus, in addition to combination with the CDA-inhibitor THU, we alternated decitabine with 5-azacytidine, to avoid the auto-dampening and instead exploit the cross-priming. Mice engrafted with syngeneic LL3 cells were assigned to treatment with (i) vehicle; (ii) THU-decitabine; (iii) THU-5-azacytidine; or (iv) THU-decitabine alternating with THU-5-azacytidine (FIG. 5A). Treatment was initiated at Day 21, after confirmation of tumor engraftment by chemi-luminescence (FIG. 5B). Repeat imaging on Day 35 demonstrated that the alternating regimen reduced tumor burden the most (FIG. 5B), and increased time-to-distress by up to 40 days versus THU-decitabine or THU-S-azacytidine alone (FIG. 5C). Analyses of tumor tissue harvested at the time-of-euthanasia suggested the greatest reduction in nuclear/cytoplasmic ratio in H&E-stained tissue sections (FIG. 5D), and also greatest Dnmt1-depletion, Myc-downregulation and p27/Cdkn1b upregulation by the alternating regimen (FIG. 5E). Besides direct effects on tumor tissues, we examined the effects of these treatments on peripheral blood immune-effector and -suppressor profiles: absolute numbers of peripheral blood CD8+ and CD4+ T-cells were significantly and similarly increased (FIG. 11A), while peripheral blood G-MDSC and M-MDSC were significantly and similarly decreased 2-4 fold, by the different treatments versus vehicle (FIG. 11A). In tumor tissues, the alternating regimen increased infiltration by CD8+ and CD4+ T-cells over THU-decitabine or THU-S-azacytidine alone (FIG. 5F). Although numbers of peripheral blood regulatory T_{reg} were not significantly changed by the treatments vs vehicle, all the treatments decreased T_{reg} by similar amounts in tumor tissue (FIG. 11B, C). All three treatment regimens similarly increased expression of cancer-germline antigens up to 12-fold (Mage-A1, Mage-A3, Mage-A4, Mage-A6) (FIG. 5G, TTD).

### Addition of Anti-Pd1 ICB to THU-Decitabine

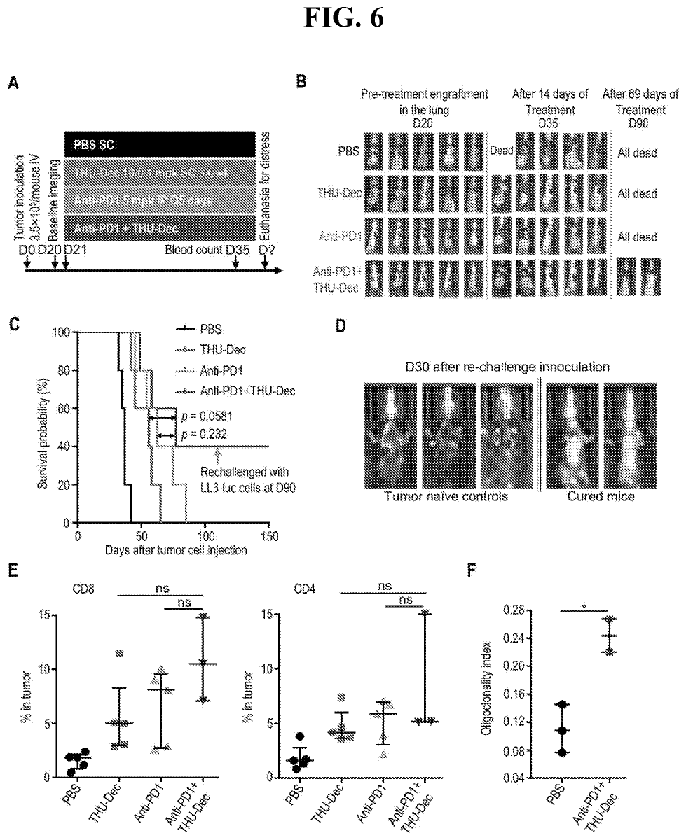

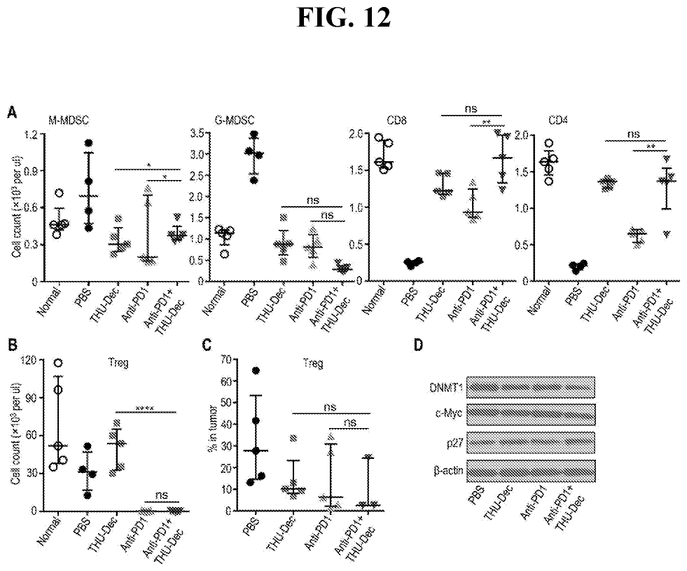

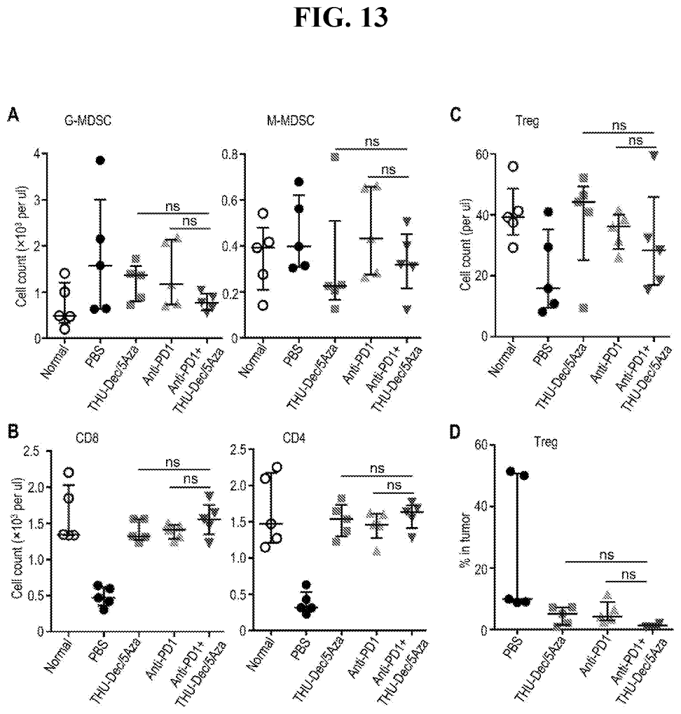

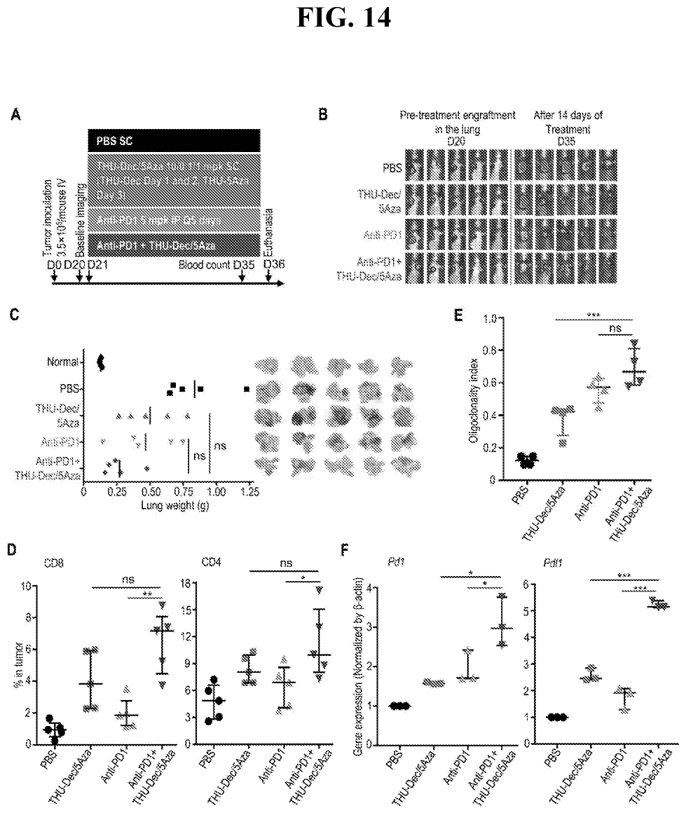

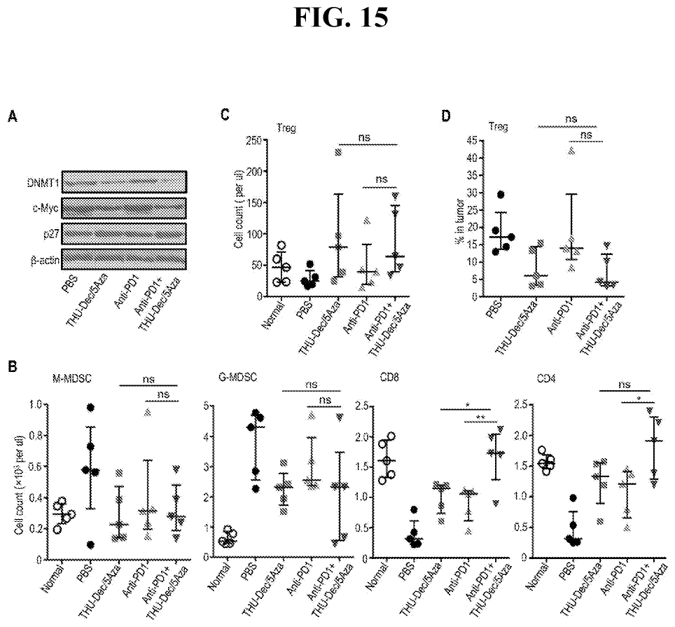

We evaluated addition of anti-Pd1 ICB to Dnmt1-depletion by THU-decitabine in the model of syngeneic disseminated lung cancer (3-5). After confirmation of tumor engraftment in the lungs using chemi-luminescent imaging on Day 20, mice were distributed to treatment with (i) vehicle; (ii) THU-decitabine alone; (iii) anti-Pd1 alone or (iv) combination THU-decitabine/anti-Pd1 (n=5/group) (FIG. 6A). Both THU-decitabine alone and anti-Pd1 alone decreased tumor burden (measured by chemiluminescence at Day 35) (FIG. 6B) and significantly increased time-to-distress versus vehicle by up to 40 days, with a slight advantage for anti-Pd1 alone over THU-decitabine alone (FIG. 6D). The combination of anti-Pd1 and THU-decitabine, however, eliminated tumor completely in 2/5 mice (these mice did not become distressed) (FIG. 6B). Indicative of an immune-component to this effect, neither of these mice engrafted lung cancer upon tail-vein reinnoculation on Day 90. The mice were sacrificed 40 days after this re-inoculation and careful examination of the lung did not reveal any tumor—while control mice inoculated at the same time succumbed to cancer by day 40 (FIG. 6C). Absolute numbers of peripheral blood CD8+ and CD4+ T-cells were significantly and similarly increased (FIG. 12A), while peripheral blood G-MDSC and M-MDSC were significantly and similarly decreased (4-fold), by the different treatments vs vehicle (FIG. 12A). Tumor infiltration by CD8+ and CD4+ T-cells was increased the most (˜10-fold) by the combination of THU-decitabine and anti-Pd1 (FIG. 6E). Peripheral blood T_{reg} were significantly decreased by anti-Pd1 and the anti-Pd1/THU-decitabine combination vs the other treatments, and T_{reg} infiltration of tumors was similarly decreased by all three treatments, but not to a significant extent (Figure S4B, C). Dnmt1-depletion, Myc-downregulation and p27/Cdkn1b upregulation was greatest with the combination treatment (FIG. 12D). T-cell oligoclonality, defined as the percentage of the T-cell population represented by the 1000 most abundant clones, and measured by RNA-sequencing to determine peripheral blood T-cell receptor (TCR) diversity, was significantly increased in mice cured by the combination treatment compared to vehicle-treated controls (FIG. 6F).

### Addition of Anti-PD1 ICB to Alternating THU-Decitabine/THU-5-Azacytidine

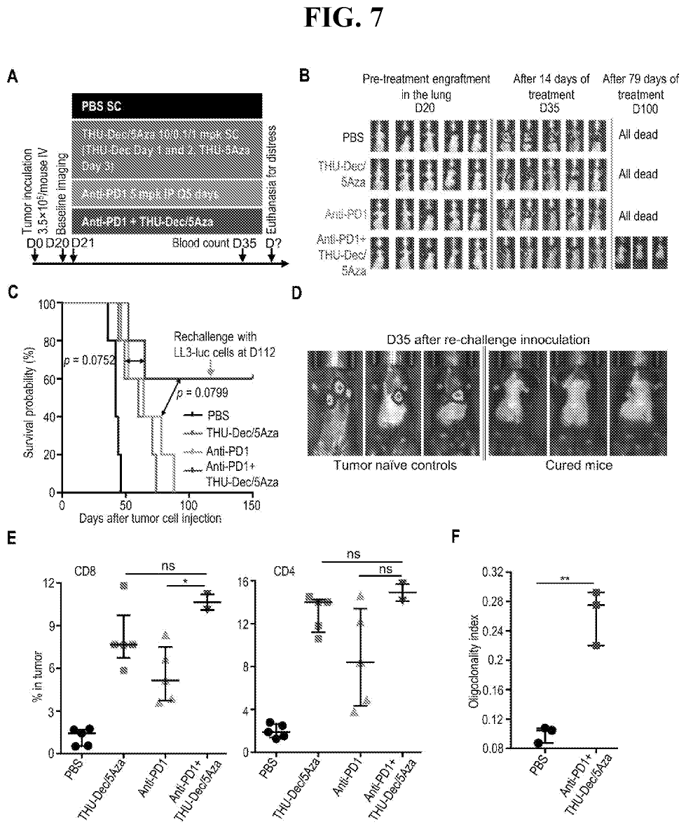

Since alternating THU-decitabine with THU-5-azacytidine more effectively depleted Dnmt1 from the lung cancer cells in vivo, we evaluated the addition of anti-Pd1 immunotherapy to this epigenetic regimen in the model of syngeneic disseminated lung cancer. After confirmation of tumor engraftment in the lungs using chemi-luminescent imaging on Day 20, mice were distributed to treatment with (i) vehicle; (ii) THU-decitabine/THU-5-azacytidine; (iii) anti-Pd1; or (iv) combination epigenetic/anti-Pd1 therapy (n=5/group) (FIG. 7A). Both the epigenetic regimen alone and anti-Pd1 alone decreased tumor burden (measured by chemiluminescence at Day 35) (FIG. 7B) and significantly increased time-to-distress vs vehicle by up to 40 days, with a slight advantage for anti-Pd1 alone over the epigenetic therapy alone (FIG. 7D). The combination of epigenetic with immunotherapy, however, eliminated tumor completely in 3/5 mice (accordingly, these mice did not become distressed) (FIG. 7B-D).

These three mice did not engraft lung cancer upon tail-vein re-challenge with the cancer cells on Day 112. The mice were sacrificed 60 days after this re-inoculation and careful examination of lungs did not reveal any tumor (FIG. 7D). Control mice inoculated at the same time succumbed to cancer by day 40 (FIG. 7C). Absolute numbers of peripheral blood CD8+ and CD4+ T-cells were significantly and similarly increased (FIG. 13). In contrast, absolute numbers of peripheral blood G-MDSC and M-MDSC were decreased by the different treatments vs vehicle (FIG. 13). Tumor infiltration by CD8+ and CD4+ T-cells was significantly increased the most (˜6-8-fold) by the epigenetic treatment alone or combination epigenetic-immunotherapy vs vehicle (FIG. 7E). T-cell oligoclonality as measured by TCR diversity analysis of peripheral blood mononuclear cells using RNA sequencing was significantly increased in mice cured by the combination treatment compared to vehicle-treated controls (FIG. 7F). Although peripheral T_{reg} numbers were unchanged by the different treatments vs vehicle (FIG. 13C), infiltration of tumor by T_{reg} was significantly and similarly decreased (FIG. 13D).

The above comparisons were after different treatment-durations dictated by different times-to-distress of the mice, and complete regression of tumors in some mice limited the ability to analyze treatment-effects on tumor tissue. The experiment was therefore repeated with sacrifice of all mice on the same treatment day 36 (after 14 days of treatment), when vehicle-treated mice became distressed (FIG. 14A). Both the epigenetic regimen alone and anti-PD1 alone decreased tumor burden by similar amounts (measured by chemiluminescence at Day 35 tumor weight at Day 36), but combination epigenetic-immunotherapy decreased the tumor burden the most by ˜70% (FIG. 14B, C). Dnmt1-depletion, Myc-downregulation and p27/Cdkn1b upregulation were also greatest with combination treatment (FIG. 15A).

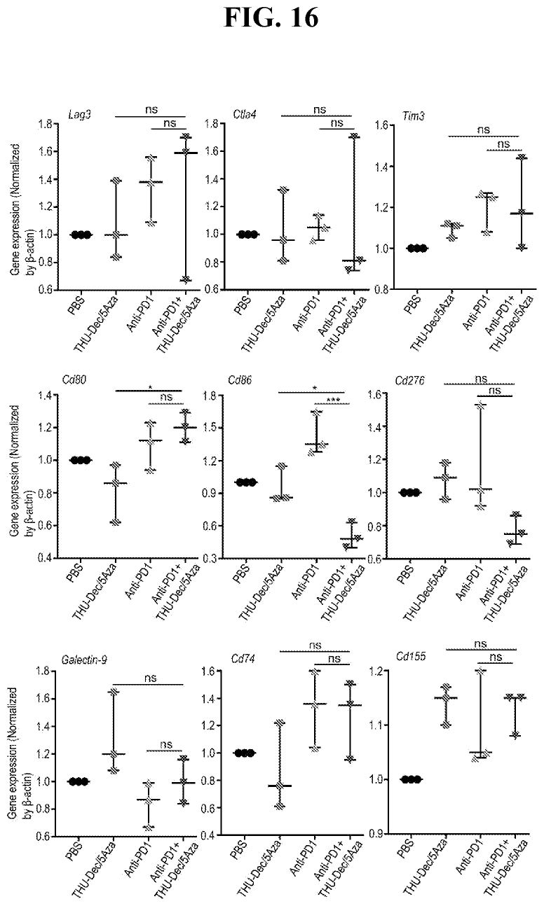

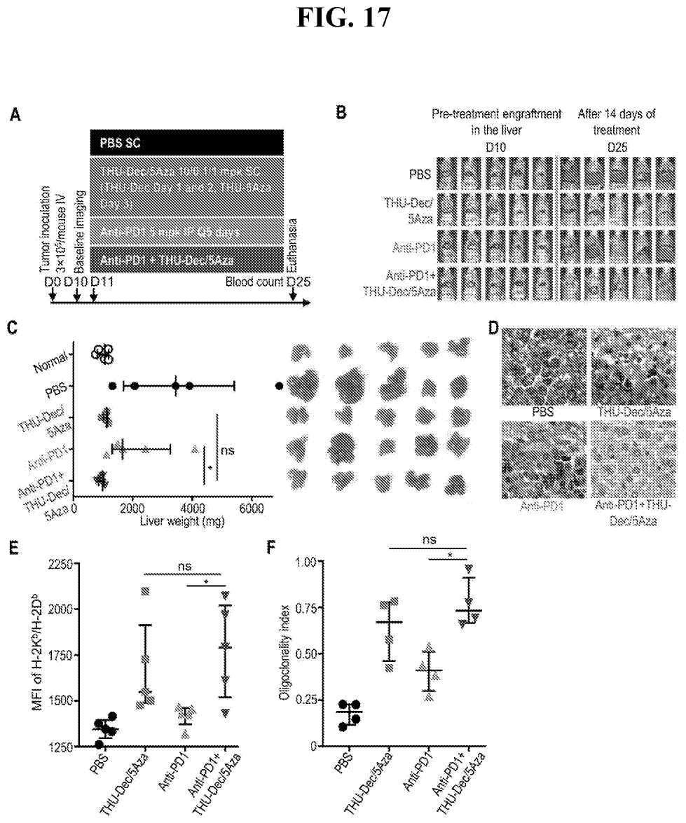

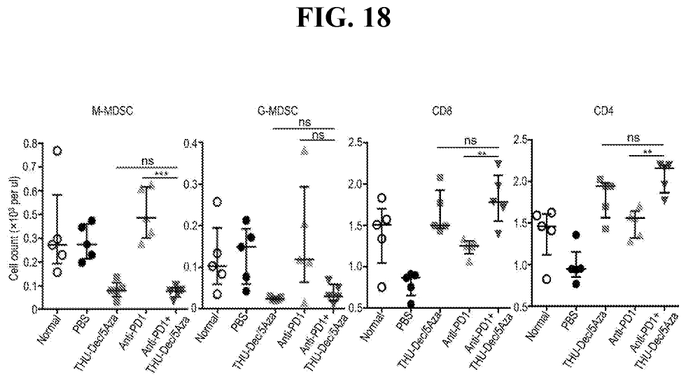

Peripheral blood CD8+ and CD4+ T-cells were significantly and similarly increased (FIG. 15B). By contrast, absolute numbers of peripheral blood G-MDSC and M-MDSC were similarly decreased by the different treatments vs vehicle (FIG. 15B). Tumor infiltration by CD8+ and CD4+ T-cells was significantly increased by the epigenetic treatment alone or combination epigenetic-immunotherapy vs vehicle (FIG. 14D). T_{reg} infiltration of tumor was decreased by all three treatments, but not to a significant extent (FIG. 15D). T-cell oligoclonality of tumor infiltrating lymphocytes (TILs) as measured by TCR diversity analysis was significantly increased by the combination treatment compared to vehicle-treated controls (FIG. 14E). The different treatments also significantly increased expression of the Pd1 check-point in TILs (isolated magnetically from tumor tissue) and Pdl1 gene expression on tumor cells, with the largest increase produced by the combination treatment (FIG. 14F). We also measured expression of several other immune checkpoints in the tumor tissue (Cd80, Cd86, Cd276, Galectin-9, Cd74, Cd155) and in TILs (Lag3, Ctla4, Tim3). Of these, only expression of Cd155 was also consistently increased in tumor tissue with the different treatments vs vehicle (FIG. 16).

### Combination ICB and THU-Decitabine/THU-5-Azacytidine in Syngeneic SCLC

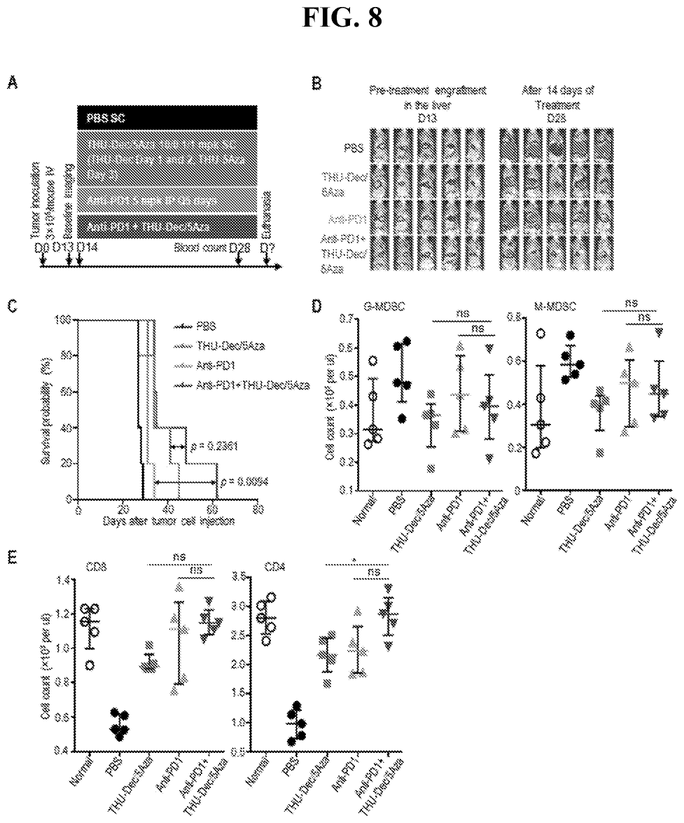

We then evaluated the epigenetic-immunotherapy treatment in the syngeneic p53-null SCLC model: F1339-luc SCLC cells were inoculated by tail-vein into syngeneic B6/129 F1 mice. After confirmation of engraftment in liver by live-imaging on Day 13, mice were distributed to treatment with (i) vehicle, (ii) THU-decitabine/THU-5-azacytidine, (iii) anti-Pd1, or (iv) combination epigenetic/immunotherapy (FIG. 8A). Anti-Pd1 treatment alone only modestly decreased tumor burden as measured by chemiluminescence imaging on Day 28 (FIG. 8B). In contrast, the epigenetic regimen alone and combination epigenetic-immunotherapy significantly and similarly decreased tumor burden (measured by chemiluminescence at Day 28) (FIG. 8B) and significantly and similarly increased time-to-distress vs vehicle, with a large advantage over anti-Pd1 alone (FIG. 8C). Absolute numbers of peripheral blood while peripheral blood CD8+ and CD4+ T-cells were significantly and similarly increased (FIG. 8E), while peripheral blood G-MDSC and M-MDSC were decreased, by the different treatments vs vehicle (FIG. 8D).

To compare different treatments administered for the same durations, the experiment was repeated but with sacrifice of all mice after 14 days of treatment (Day 25 when vehicle-treated mice became distressed) (FIG. 17A). Anti-Pd1 treatment alone only modestly decreased tumor burden as measured by chemiluminescence imaging (FIG. 17B) and by liver weight (FIG. 17C). By contrast, the epigenetic regimen alone and combination epigenetic-immunotherapy significantly and similarly decreased tumor burden (FIG. 17B, C), with a prominent advantage over anti-Pd1 alone (FIG. 17B, C). Tumor tissue histological sections showed decreased nuclear-cytoplasmic ratio and increased necrosis with the epigenetic regimen alone or combination epigenetic-immunotherapy vs vehicle or anti-Pd1 alone (FIG. 17D). The epigenetic treatment alone and combination epigenetic-immunotherapy also produced significant increases in antigen-presenting H-2K^{b}/H-2D^{b} molecules (FIG. 17E). Oligoclonality of TILs, measured by RNA-sequencing of the T-cell receptor, was also most prominent with epigenetic treatment alone and combination epigenetic-immunotherapy vs vehicle or anti-Pd1 alone (FIG. 17F). Absolute numbers of peripheral blood CD8+ and CD4+ T-cells were significantly and similarly increased (FIG. 18), while peripheral blood G-MDSC and M-MDSC were decreased, by the different treatments vs vehicle (FIG. 18).

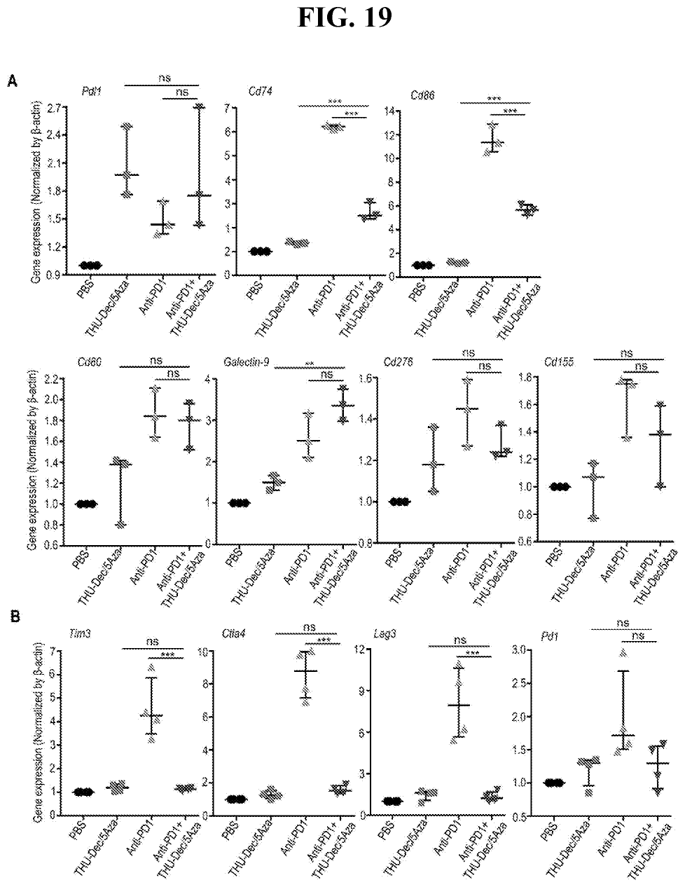

To investigate why anti-Pd1 treatment did not add much benefit to the epigenetic treatment in the syngeneic SCLC model (contrasting with the results in the NSCLC models) we measured expression of several immune checkpoint pathway genes in SCLC tumor tissue (Pdl1, Cd80, Cd86, Cd276, Galectin-9, Cd74, Cd155), and in TILs isolated magnetically from the tumor tissue (Pd1, Lag3, Ctla4, Tim3) after the 14 days of treatment. Treatment of SCLC by anti-Pd1 was linked with several-fold increases in expression of multiple immune-checkpoints other than Pd1/Pdl1, namely Cd74, Cd80, Galectin 9, Tim3, Ctla4 and Lag3 (FIG. 19).

The pyrimidine nucleoside analog pro-drugs decitabine and 5-azacytidine traverse pyrimidine metabolism, each by their own path, and convert into the nucleotide Aza-dCTP to deplete the epigenetic target DNMT1. We found here that both pro-drugs induce rapid changes in expression by lung cancer cells of key pyrimidine metabolism enzymes DCK, UCK2, CDA and CAD in ways expected to impede uptake, nucleotide conversion and DNMT1-depletion by the applied agent, but enhance DNMT1-depletion if the sister agent is applied and CDA is inhibited. Specifically, decitabine upregulated UCK2 and CDA ˜3-fold within 72-96 hours—UCK2 salvages cytidine, enabling cells to bypass the deoxycytidine analog decitabine. CDA deaminates decitabine into uridine moiety counterparts that do not deplete DNMT1. On the other hand, 5-azacytidine upregulated DCK ˜3-fold and CAD ˜2-fold—DCK salvages deoxycytidine, enabling cells to bypass the cytidine analog 5-azacytidine; CAD bypasses 5-azacytidine (and decitabine) by generating cytidines de novo. Of these enzyme expression shifts, rapid upregulation of CDA by decitabine, and of DCK by 5-azacytidine, has been reported previously. Decitabine upregulated CDA in leukemia and solid tumor cells by 6 to 1000-fold within 96 hours (30, 31), while 5-azacytidine upregulated DCK in leukemia cells by ˜30% within 48 hours (32).

Why do decitabine and 5-azacytidine rapidly and distinctly change pyrimidine metabolism enzyme expression? Decitabine and 5-azacytidine drive levels of dCTP, a key metabolic end-product that regulates nodes in the pyrimidine metabolism network (16), in opposite directions—decitabine increases dCTP and 5-azacytidine decreases it. After phosphorylation by DCK and deamination by DCTD, decitabine inhibits TYMS (˜10% of decitabine is expected to traverse this route)—this reduces cellular dTTP by >50% but increases dCTP, because lower dTTP dis-inhibits ribonucleotide reductase to generate more dCTP (ribonucleotide reductase conversion of CDP into dCDP is allosterically controlled by dTTP) (17-19). By contrast, 5-azacytidine decreases dCTP by partially inhibiting ribonucleotide reductase (di-phosphorylated 5-azacytidine is a substrate for ribonucleotide reductase) (21). In summary, while the present invention is not limited to any particular mechanism and an understanding of the mechanism is not necessary to understand or practice the invention, it is believed that opposite effects of decitabine vs 5-azacytidine on cellular dCTP amounts (17-19, 21) plausibly drive the diverging adaptive responses of pyrimidine metabolism to the individual pro-drugs. Notably, direct regulation of eukaryotic gene transcription by metabolites has been described (33).

DNMT1 is highly validated scientifically as a molecular target for p53-independent (non-cytotoxic) cytoreduction of both solid tumor and myeloid malignancies, but solid tumor clinical trial results with decitabine or 5-azacytidine have been largely disappointing, especially relative to meaningful results and regulatory approval to treat myeloid malignancies (7). One basis for this discrepancy between myeloid and solid tumor malignancies, that also explains why decitabine and 5-azacytidine are not routinely administered by the oral route, is trivial distribution through high-CDA solid tissues such as the intestines and liver (9, 34). CDA rapidly deaminates both decitabine and 5-azacytidine into non-DNMT1-depleting but potentially cytotoxic uridine nucleoside analogs, shortening in vivo plasma half-lives to ˜15 minutes vs 9-16 hours in vitro at 37° C. (9, 35-38). Accordingly, addition of the CDA-inhibitor THU increases by ˜10-fold the oral bioavailability and plasma half-lives of decitabine and 5-azacytidine, thus enabling tumor cytoreductions even in CDA-rich solid tissue organs (9, 10, 34, 39). CDA upregulation within cancer cells has also been reported by several groups to be a mechanism by which cancer cells resist cytidine analog drugs (reviewed in (9)). Thus, there are both extra-cellular and intra-cellular rationales for combining decitabine or 5-azacytidine with the CDA-inhibitor THU, and THU consistently increased the efficacy of these drugs ˜2-fold, without bone marrow suppression, in our in vivo studies of murine lung cancer.

Another significant pharmacologic barrier to decitabine and 5-azacytidine activity in solid vs myeloid malignancies is several-fold lower baseline expression in solid tumors of DCK and UCK2, which as discussed earlier, rate-limit respectively decitabine and 5-azacytidine uptake rates and intra-cellular half-lives (15). Decitabine induction of UCK2, and 5-azacytidine induction of DCK is a practical method to attack this barrier—THU-decitabine alternating with THU-5-azacytidine significantly increased DNMT1-depletion and therapeutic benefit over THU-decitabine or THU-5-azacytidine alone in the murine models of lung cancer, again without causing myelosuppression (the myeloid compartment consists of waves of cells that proliferate transiently then terminally differentiate, likely creating less opportunity for auto-dampening and cross-priming).

DNMT1-depletion has also been extensively shown to increase immune-recognition of cancers by upregulating genes that mediate antigen presentation, type I and type II interferon signaling, and viral defense (3), and hence responses to ICB (40). The pre-clinical evidence is such that there are >10 clinical trials evaluating the combination of parenteral decitabine or 5-azacytidine with ICB (4, 5, 41-43). Unfortunately, the same pharmacologic barriers to previous attempts at clinical translation also threaten these trials. In the present Example, the DNMT1-depleting regimens designed to overcome these barriers produced greater upregulation of antigen presenting MHC class I molecules (MHC I H-2K^{b}/H-2D^{b}) and cancer-germline antigens (MageA1-A4) (that are displayed in MHC class I). Also encouraging, there was greater infiltration of tumor by cytotoxic T-cells and greater shifts towards T cell oligoclonality, and activation of T-cells with increased expression of IFN-γ, perform and granzyme B. The non-cytotoxic mechanism of action preserved immune-effectors and made possible long-term, chronic therapy, shown also previously in pre-clinical murine models of acute myeloid leukemia (9, 26, 29) and non-human primates (10), and clinically in patients with myeloid malignancies and non-malignant disease treated with non-cytotoxic regimens of parenteral decitabine, oral THU-decitabine or oral THU-5-azacytidine (27, 34, 39, 44). Since clinical data have suggested that reduction of immunosuppressive regulatory immune cells by DNMT1-depleting drugs may be as important as stimulating effector cell-mediated antitumor immunity (45), it is notable that the pharmacologically optimized DNMT1-depleting regimens were also superior to decitabine or 5-azacytidine alone in decreasing numbers of MDSCs and regulatory T-cells. Accordingly, when combined with ICB, complete tumor regressions were produced in some animals, and these animals even resisted fresh tumor cells inoculated by repeat tail-vein injection, a remarkable benefit that implied generation of memory T-cell responses against tumor antigens (46).

Although the non-cytotoxic Dnmt1-depleting regimen significantly cytoreduced p53-null SCLC, increased tumor infiltration by immune-effectors and suppressed MDSC, the addition of ICB using anti-Pd1 did not add much more benefit. While the present invention is not limited to any particular mechanism, one possible explanation is that SCLC suppresses or ‘checks’ immune attack using as dominant pathways molecules other than Pdl1. Several immune checkpoints other than Pd1 were significantly upregulated in the SCLC tumors treated with anti-Pd1, including the Tim3, Ctla-4 or Lag3 checkpoint pathways.

Scientific rationale for decitabine and 5-azacytidine combinations with immune-therapies is compelling enough that >10 solid tumor clinical trials are underway or completed, despite disappointing results from preceding trials (5, 7). We propose that persistent translation difficulties could reflect pharmacology problems, such as disadvantageous baseline expression patterns of the key pyrimidine metabolism enzymes CDA, DCK and UCK2 in solid tumors such as lung cancer, that are further exacerbated by adaptive shifts in expression in response to decitabine or 5-azacytidine.

### Materials and Methods

### Study Design

The objectives of this study were to identify potential mechanisms underlying failure of clinical trials combining DNMT1-depleting pyrimidine nucleoside analog pro-drugs with ICB, and to evaluate mechanism-based solutions. The central hypothesis was that the regulated network structure of pyrimidine metabolism reacts to automatically dampen activity of administered pyrimidine nucleoside analogs, and that these adaptive responses of metabolism can be anticipated and harnessed to improve response instead. The experimental approach first validated the key pyrimidine metabolism enzymes mediating nucleoside analog pro-drug activity, by correlating in vitro drug-sensitivity (growth-inhibition) in the NCI60 panel of cancer cell lines with expression of pyrimidine metabolism enzymes. Then, how expression of key pyrimidine metabolism enzymes changes upon cancer cell exposure to nucleoside analogs versus natural pyrimidine nucleosides was determined using at least three independent biological replicates. Two in vivo models of aggressive, disseminated syngeneic murine lung cancer were then used to evaluate candidate solutions to exploit adaptive responses of the pyrimidine metabolism network, both alone and in combination with ICB. Mice cured by combination epigenetic-immunotherapy were re-challenged by tail-vein inoculation of cancer cells to evaluate if the mechanism of cure incorporated immune memory against the cancer. Live imaging was used to verify tumor engraftment prior to initiation of therapy, with mice distributed to treatment groups to balance baseline tumor burden. Each in vivo experiment was powered to show statistically significant results between treatment groups, possible with five mice per treatment group because of treatment-effect sizes. Eight independent in vivo treatment experiments were conducted, with >180 mice treated in total. All mice were female, to avoid confounding of interpretation by sex-differences in pro-drug metabolism. All in vivo experiments validated that DNMT1-depleting therapy preserved immune-effectors and increased tumor immune-recognition/infiltration, using standard methods. That non-cytotoxic DNMT1-depletion can cytoreduce chemorefractory p53-null cancer was verified both in vitro and in vivo.

### Cell Lines and Culture

LL3 Lewis lung cancer cells were employed. F1339 small cell lung cancer cells were also employed. Both cell lines were maintained in RPMI1640 supplemented with 10% fetal bovine serum (FBS, Gibco), 100 units/ml penicillin, 100 μg/ml streptomycin (Gibco), and cultured at 37° C. in 5% CO₂. These cell lines are not in the database of commonly misidentified cell lines (ICLAC and NCBI Biosample). The cell lines regularly tested negative for Mycoplasma contamination.

### Mice

Female C57BL/6, B6/129SF1/J and NSG mice were purchased from Jackson Lab (Bar Harbor, Me., USA). Mice were inoculated with tumor cells at age 4-6 week old. Mice were maintained under specific pathogen-free conditions, with free access to food and water. All procedures were performed in compliance with the legislation on the use and care of laboratory animals, and according to protocols (2013-1137 and 2017-1863) approved by the Institutional Animal Care and Use Committee (IACUC) of Cleveland Clinic.

### Syngeneic Tumor Models

C57BL/6 mice were tail-vein inoculated with 0.35×10⁶ LL3 cells, and B6/129SF1/J mice with 0.3×10⁶ F1339 cells. Assignment to different treatments was after documentation of tumor engraftment by live imaging. Peripheral blood monitoring on-treatment was by tail-vein phlebotomy. Mice were euthanized for signs of distress as defined in the Animal Protocols, and blood and tumor were collected for further analyses. Apparently cured mice were re-challenged with fresh tumor cells (0.5×10⁶) inoculated by tail vein.

### Patient-Derived Xenotransplant Model

Primary tumor and pleural fluid was collected from a lung cancer patient after written informed consent on IRB-approved protocol (07-267). Six-week-old female NSG mice were inoculated in the right flank with 5×10⁶ tumor cells. On day 3 after tumor inoculation, 5×10⁶ human PBMCs from the same patient were inoculated via tail-vein. Mice were distributed such that baseline tumor volume was similar between treatment groups. Tumor volume was recorded twice a week and the experiment was terminated when the tumor volume in vehicle treated mice reached 2000 mm³.

### Flow Cytometry Analysis

Peripheral blood mononuclear cells were stained with CD3, CD4, CD8, CD11b, Ly6C, Ly6G antibodies (BD Biosciences). The stained cells were then fixed with fixation buffer (eBioscience). The FOXP3 staining buffer set (eBioscience) was used for intranuclear staining. For flow cytometry analyses of tumor tissue, tumor tissue was first digested by cutting into small fragments then incubated with mouse or human tumor enzyme cocktail per the manufacturer's protocol with gentlMACS tubes and the gentleMACS dissociator (Miltenyi). After filtering through a 70-μm strainer (Thermo Fisher Scientific), the single-cell tumor suspension was centrifuged on a 30% percoll gradient over a 70% percoll gradient to enrich for mononuclear cells and remove debris. Cells were stimulated with PMA/ionomyocin in the presence of Golgi stop and Monesin (eBioscience) for 4 hours, then washed with PBS and stained with Live/Dead stain (Life Technologies) and antibodies specific to cell surface markers CD3, CD4 and CD8 (BioLegend). After fixation-permeabilization (Fixation/Permeabilization buffer, eBioscience), cells were stained with antibodies specific to IFNr, FOXP3, granzyme B and perforin (BioLegend) or with the isotype control antibodies. Flow cytometry analysis was performed on BD LSRFortessa and data was analyzed by FlowJo V10.

### Quantitative Polymerase Chain Reaction

RNA was extracted using Trizol reagent (Invitrogen) before the generation of complementary DNA using the iScript™ cDNA Synthesis Kit (Bio-rad). Quantitative PCR was run on StepOnePlus Real-Time PCR System (Applied Biosystems), using comparative Cₜ value method to quantify the expression of target genes in different samples. Gene expression was normalized to the housekeeping gene β-actin.

### Western Blot

Tumor cells were lysed with RIPA lysis buffer and protein concentrations determined by BCA protein assay kit (Thermo Fisher Scientific). Samples with equal quantity (40 μg) of total protein were mixed with 4× loading buffer and 10× sample reducing reagent (Thermo Fisher Scientific), subjected to electrophoresis on a 12% (v/v) SDS-polyacrylamide gel, then transferred onto polyvinylidene fluoride membranes. After blocking with 5% dried skimmed milk, the membranes were washed three times and incubated with primary antibodies at 4° C. overnight. After washing, the membranes were further incubated with corresponding horseradish peroxidase-conjugated secondary antibodies. The membranes were then treated with Pierce™ ECL substrates (Thermo Scientific) then visualized using X-ray film.

### Histological Analysis

Tumors were fixed in 10% buffered formalin phosphate (Thermo Fisher Scientific) for 12 hours and embedded in paraffin. Sections were stained using H&E.

### T-Cell Receptor (TCR) Sequencing (T-Cell Oligoclonality Analyses)

RNA was isolated from PBMC or CD8+ TILs using the AllPrep RNA Mini Kit (Qiagen)-200 ng of total RNA was used to construct TCR alpha and beta chain (a/b) libraries using the SMARTer Mouse TCR a/b Profiling Kit (Takara) per manufacturer's instruction. Samples were pooled to a final concentration of 4 nM and then the pooled libraries were further diluted to a final concentration of 13.5 μM including a 7% PhiX Control v3 (Illumina) spike-in. Sequencing was performed on an Illumina MiSeq sequencer (Illumina) using the 600-cycle MiSeq Reagent Kit V3 (Illumina) with paired-end, 2×300 base pair reads. The data was analyzed with MiXCR 1.1.0 (Illumina).

### Statistical Analysis

Statistical analysis was performed with Prism 7.0 software (GraphPad, San Diego, Calif.). Survival differences among the treatment groups were analyzed by the Kaplan-Meier method and p values were calculated with log-rank test. Two-sided Mann-Whitney U test was used to compare medians and a two-sided unpaired t test to compare means. Bonferroni's correction to p<0.05 was used to determine statistical significance.

### Example 2

### Human Cancer Treatment with Staggered Decitabine and 5-Azacytidine

This Example describes various treatment protocols that could be used to treat human patients with cancer, such as pancreatic cancer.

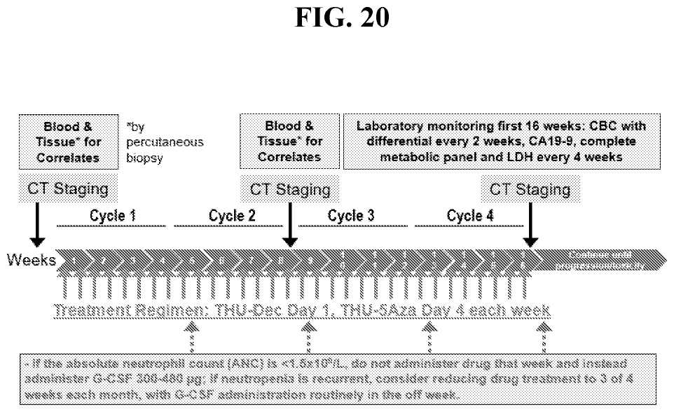

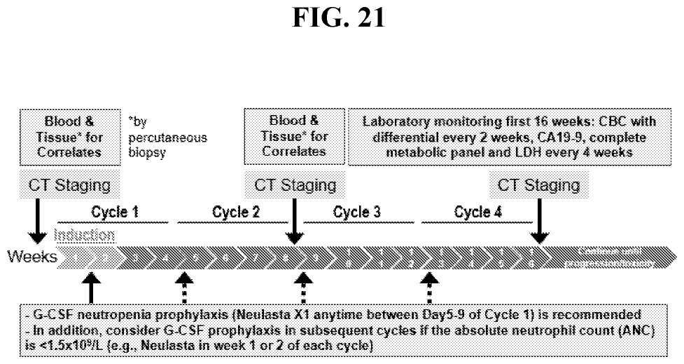

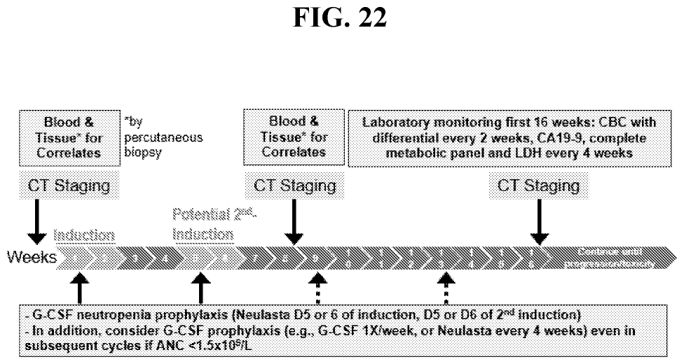

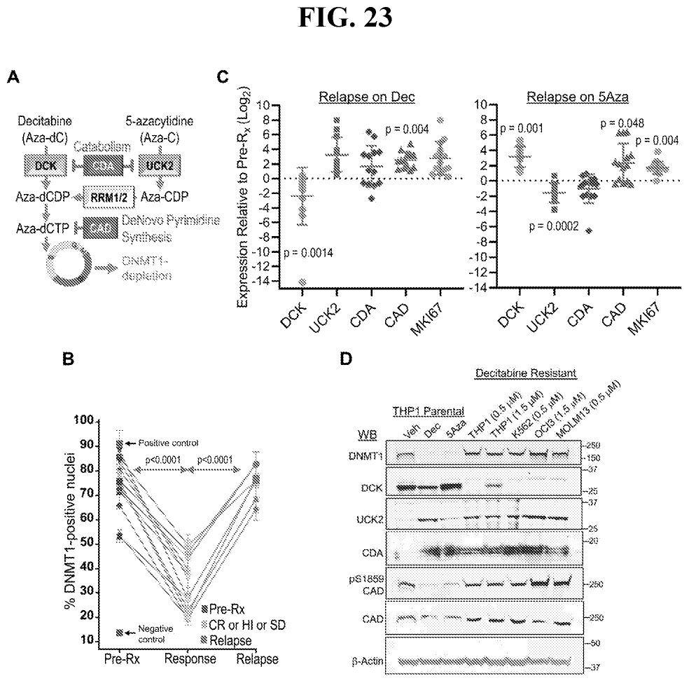

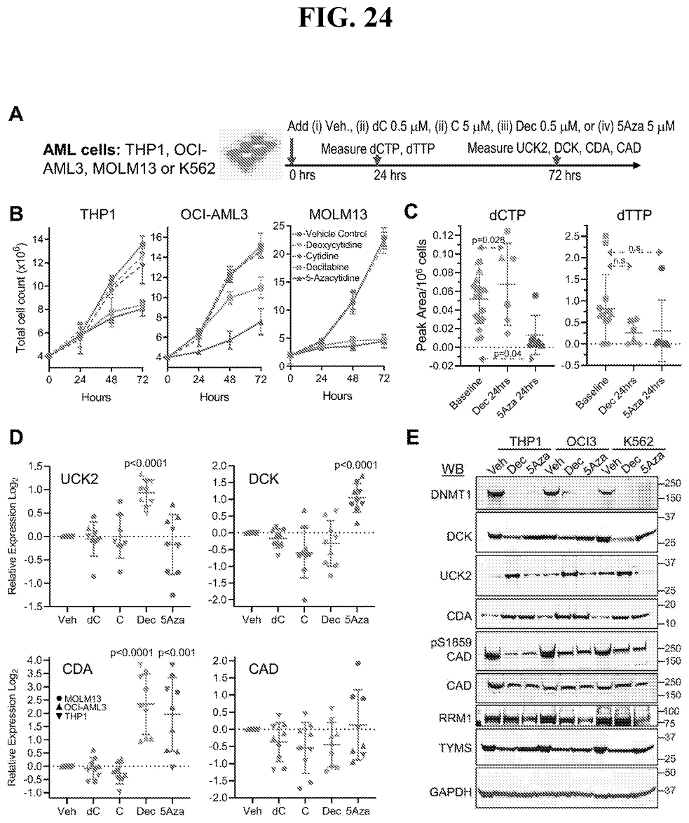

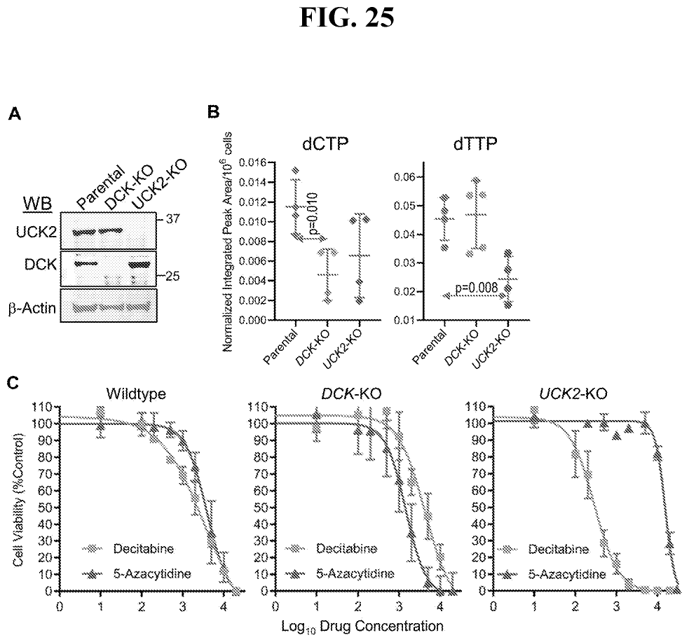

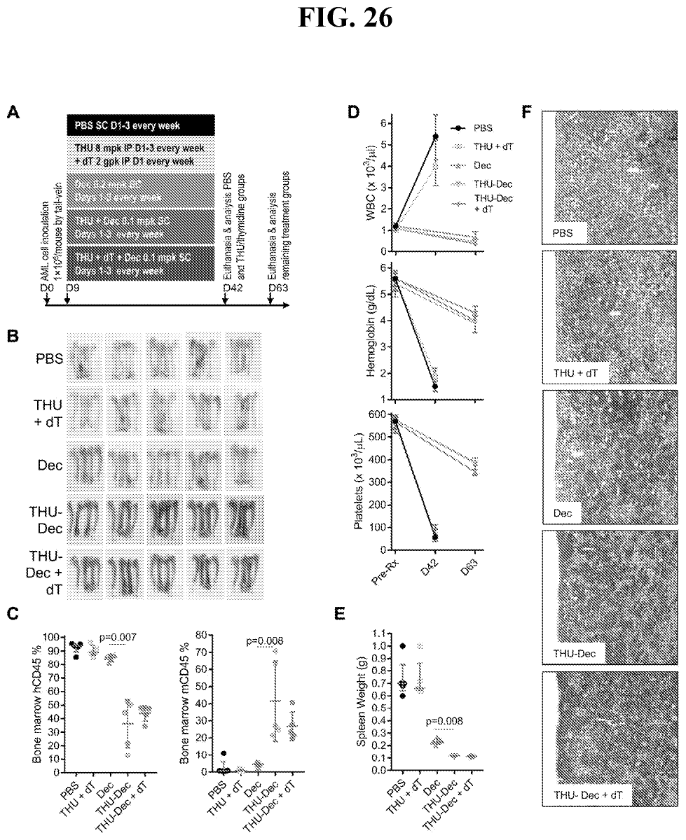

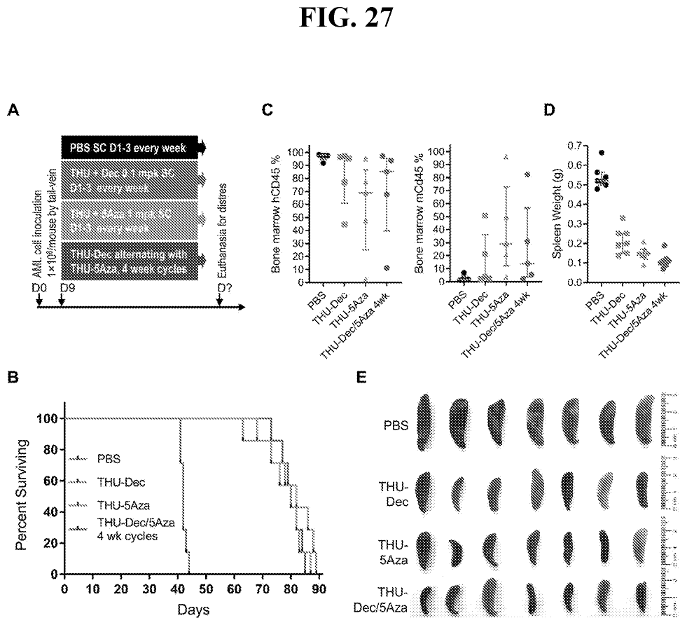

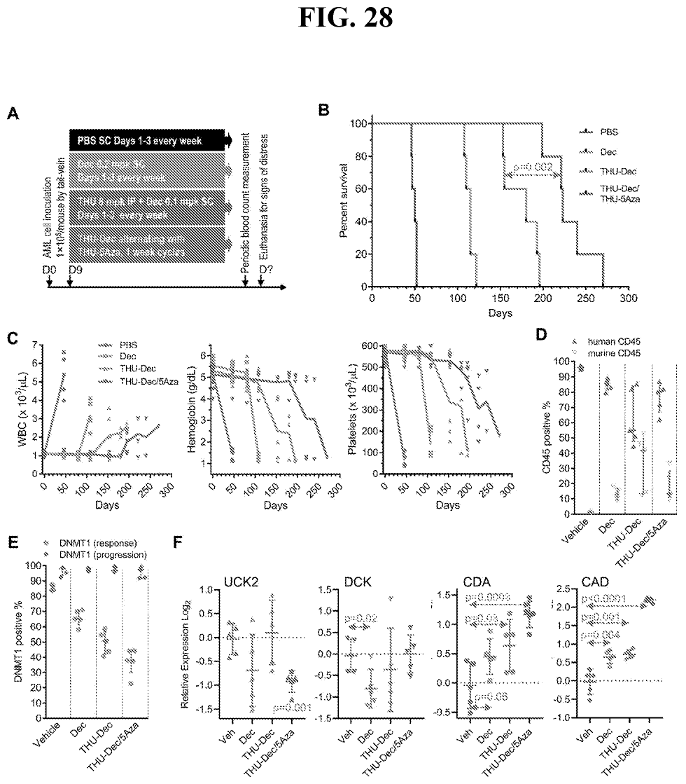

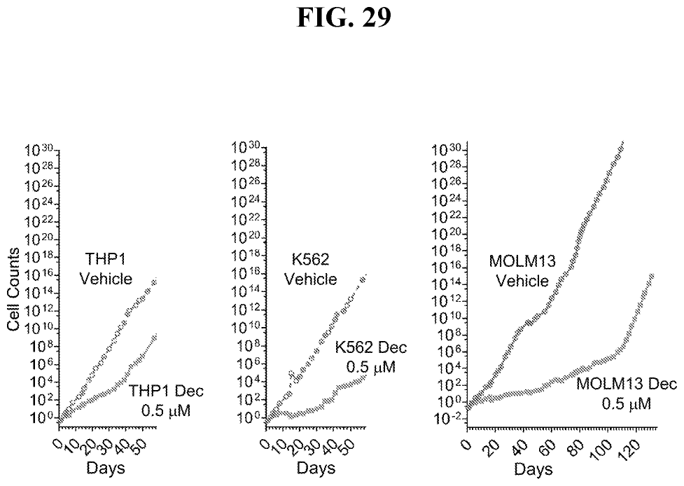

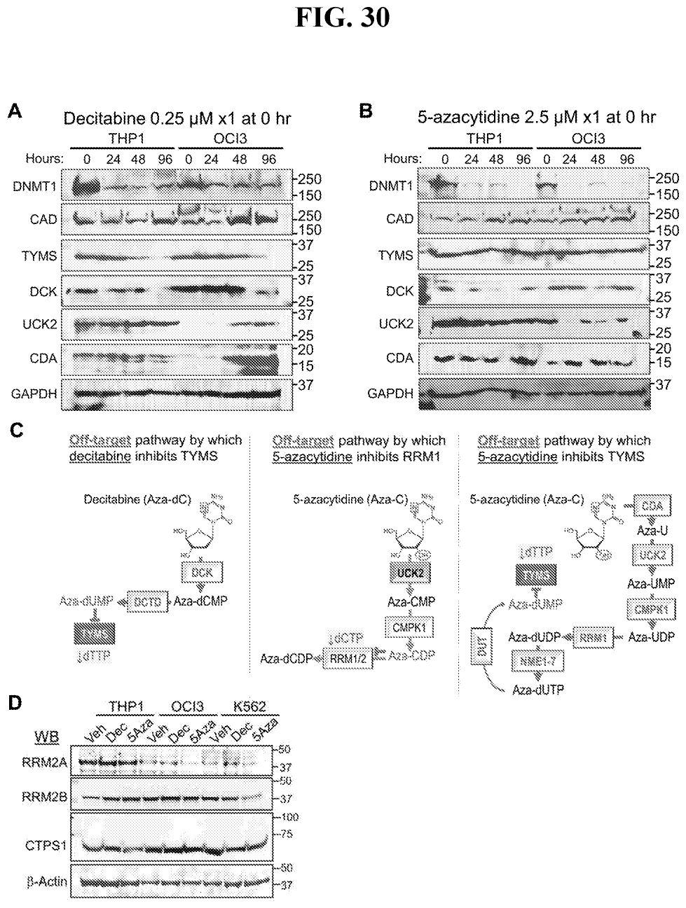

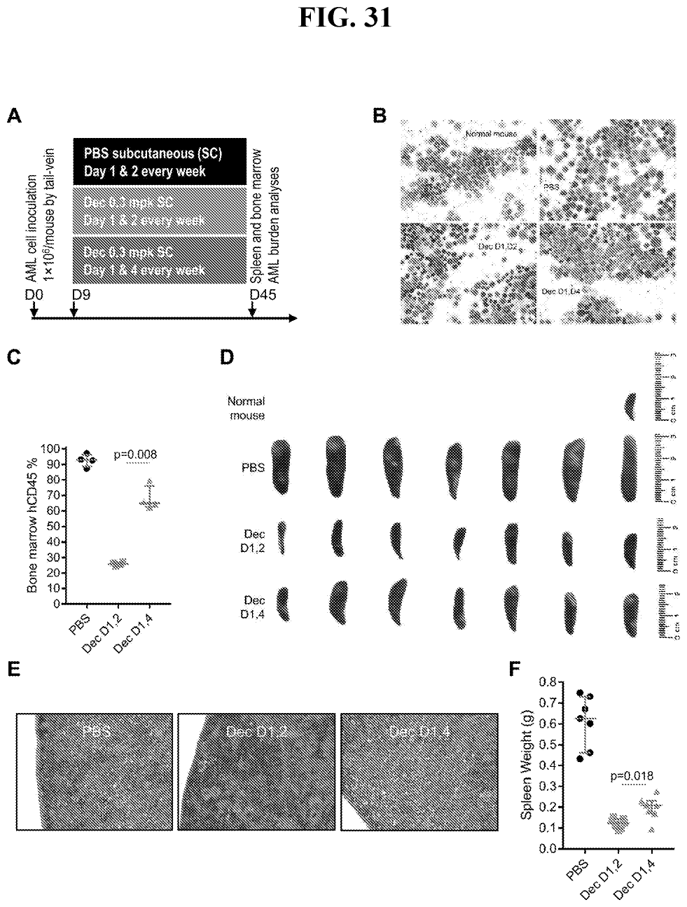

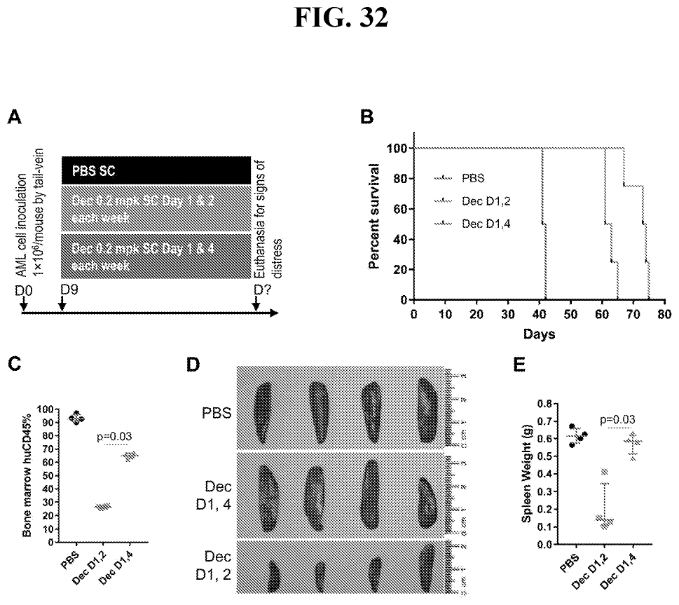

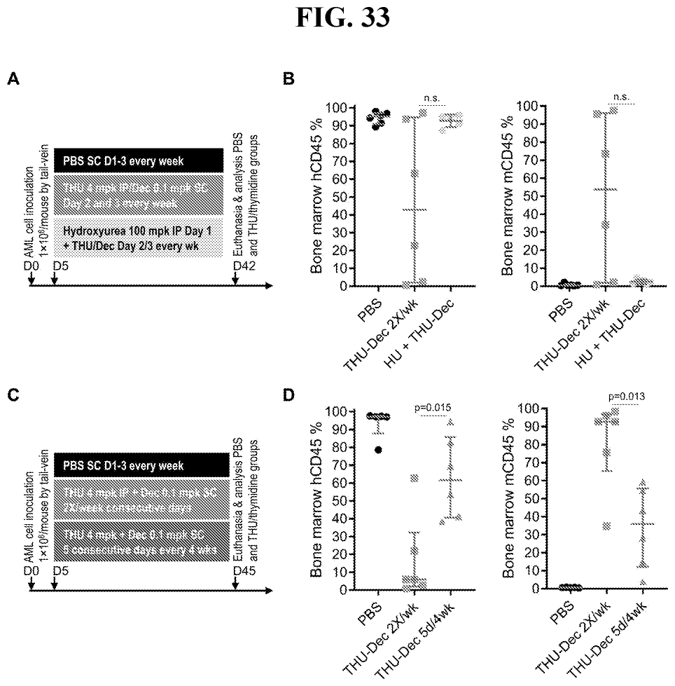

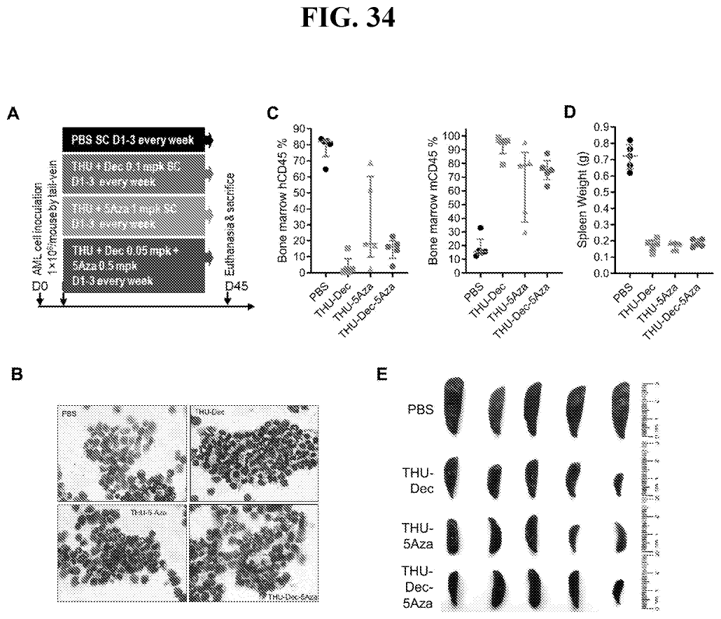

FIG. 20 describes a first exemplary treatment staggered decitabine and 5-azacytidine weekly treatment protocol for a human patient with cancer over a treatment period of multiple weeks. This protocol includes the possibility of skipping treatment a given week if absolute neutrophil count (ANC) is too low. In such instances, the patient can be administered G-CSF.

FIG. 21 describes a second exemplary treatment staggered decitabine and 5-azacytidine weekly treatment protocol for a human patient with cancer over a treatment period of multiple weeks. In this protocol, there is an induction period where the patient is administered THU and then decitabine 60 minutes later for five consecutive days, and then G-CSF (NEULASTA neutropenia prophylaxis) anytime between days 5-9.

FIG. 22 describes a third exemplary treatment staggered decitabine and 5-azacytidine weekly treatment protocol for a human patient with cancer over a treatment period of multiple weeks. This protocol has a first induction protocol and potential second induction protocol as described in FIG. 22.

A fourth exemplary protocol is as follows. THU-Decitabine administered to a human with cancer on day 1, THU-5-Azactidiine administered on day 4, of every week for at least four weeks. All drugs are oral. THU dose is ˜10 mg/kg; capsules are 250 mg each. Decitabine dose is ˜0.16 mg/kg; capsules are 5 mg each. 5-Azacitidine dose is 1.6 mg/kg; capsules are 50 mg each. The following dosing schedule is used for starting doses: i) weight ≤45 kg: 2 capsules of THU followed 60-360 minutes later by 1 capsule of decitabine (on Day 1) or 1 capsule of 5-azacytidine (on Day 4) (e.g., THU at breakfast, Decitabine or 5-azacytidine at lunch); ii) weight 45-80 kg: 3 capsules of THU followed 60-360 minutes later by 2 capsules of decitabine (on Day 1) or 2 capsules of 5-azacytidine (on Day 4); and iii) weight 81 kg or higher: 4 capsules of THU followed 60-360 minutes later by 3 capsules of decitabine (on Day 1) or 3 capsules of 5-azacytidine (on Day 4).

### Example 3

### Timed Decitabine and 5-Azacytidine Treatment

### Methods

Study approvals. Bone marrow samples for research were obtained from patients with AML (Acute myeloid leukemia) with written informed consent on a study protocol approved by the Cleveland Clinic Institutional Review Board (Cleveland, Ohio). Murine experiments were approved by the Cleveland Clinic Institutional Animal Care and Use Committee (Cleveland, Ohio).
Sources of cell lines and animals. AML cell lines OCI-AML3 were purchased from DSMZ (Braunschweig, Germany), and THP1, K562 and MOLM13 cell lines were purchased from ATCC (Manassas, Va.). The AML cell lines, including those selected for resistance to decitabine, were authenticated (Genetica cell line testing, Burlington, N.C.). DCK and UCK2 knock-out leukemia (HAP1) cells were engineered via Horizon Discoveries (Cambridge, United Kingdom). Primary AML cells for inoculation into NSG mice were collected with written informed consent on Cleveland Clinic Institutional Review Board approved protocol 5024. NSG mice were purchased from Jackson Laboratories (Bar Harbor, Me.).
DNMT1 Immuno-detection and quantitation: Immunohistochemistry (IHC) was performed on decalcified and formalin-fixed paraffin embedded bone marrow biopsy sections (4 μm) and on positive and negative controls (parental and DNMT1-KO HCT116 cells). Antibodies used were mouse polyclonal anti-Dnmt1 (Abcam #ab19905, Cambridge, Mass.), 1:200 dilution for 32 minutes at room temperature, performed with Ventana Discovery using OmniMap detection and a high pH tris-based buffer (Cell Conditioning 1, Ventana #950-124). Nuclei positive for DNMT1 were identified and quantified in high resolution, large field-of-view images per ImageIQ algorithms (Image IQ Inc., Cleveland, Ohio) after segmentation of images and subtraction of bone as has been previously described⁴.

DNMT1-protein measurement by flow cytometry was performed as previously described⁴⁰ using unlabeled anti-Dnmt1 antibody [EPR 3522] (0.0625 μg/test; Abcam; catalog no. ab92314) as the primary antibody

DNA isolation, reverse transcription (RT) and real-time PCR. As previously described⁵⁹. Primer sequences were:

| Gene | Primer (Forward) | Primer (Reverse) |
|---|---|---|
| CAD | 5′-CTGACTTCTACACTGAGC | 5′-CACGCATTGACAGGTTAA |
| | ATGG-3′ SEQ ID NO: 1 | TCAC-3′ SEQ ID NO: 2 |
| CDA | 5′-AAGGGTACAAGGATTTCA | 5′-ACAATATACGTACCATCC |
| | GGG-3′ SEQ ID NO: 3 | GGC-3′ SEQ ID NO: 4 |
| DCK | 5′-AAGCTGCCCGTCTTTCT | 5′-ACCACTTCCCAATCTTCA |
| | C-3′ SEQ ID NO: 5 | CAC-3′ SEQ ID NO: 6 |
| UCK2 | 5′-ATCCCCGTGTATGACTTT | 5′-CTTCATCTGGAACAGGTC |
| | GTC-3′ SEQ ID NO: 7 | TCG-3′ SEQ ID NO: 8 |
| GAPDH | 5′-ACATCGCTCAGACACCAT | 5′-TGTAGTTGAGGTCAATGA |
| | G-3′ SEQ ID NO: 9 | AGGG-3′ SEQ ID NO: 10 |

1D SDS-polyacrylamide gel electrophoresis and Western blot analysis. Were performed as we previously described⁵⁹. Antibodies used were:

| | | |
|---|---|---|
| Catalogue# | Name | Company |

Primary Antibodies Used

| | | |
|---|---|---|
| ab13537 | DNMT1 antibody | Abcam |
| sc-393098 | dCK Antibody (H-5), Mouse | Santa Cruz |
| ab104731 | Anti-UCK2 antibody, Rabbit | ABCAM |
| 12662 | Phospho-CAD (Ser1859) | Cell Signaling |
| 11933 | CAD Antibody | Cell Signaling |
| GTX108663 | DCTD antibody | GeneTex |
| sc-390945 | TYMS Antibody (C-5) | Santa Cruz |
| sc-377415 | RRM1 Antibody (A-10) | Santa Cruz |
| sc-398294 | RRM2A Antibody (A-5) | Santa Cruz |
| PRS2383-100UG | Anti-P53R2 antibody (RRM2A) | Sigma-Aldrich |
| ab82347 | Anti-CDA antibody | ABCAM |
| sc-374015 | Lamin B1 Antibody (B-10) Alexa | SCBT |
| AF488 | Fluor ® 488 | |
| sc-47724 AF647 | GAPDH Antibody (0411) Alexa | SCBT |
| | Fluor ® 647 | |
| F3022-.2mL | Actin-FITC | Sigma-Aldrich |
| A304-543A | Rabbit anti-CTPS1 Antibody | Bethyl Lab |

Secondary Antibodies Used

| | | |
|---|---|---|
| A32735 | Goat anti-Rabbit IgG (H + L) | Invitrogen |
| | Highly Cross-Adsorbed Secondary | |
| | Antibody, Alexa Fluor Plus 800 | |
| A32730 | Goat anti-Mouse IgG (H + L) | Invitrogen |
| | Highly Cross-Adsorbed Secondary | |
| | Antibody, Alexa Fluor Plus 800 | |
| 12004158 | StarBright ™ Blue 700 Goat Anti- | Biorad |
| | Mouse IgG, 400 μl | |
| 12004161 | StarBright ™ Blue 700 Goat Anti- | Biorad |
| | Rabbit IgG, 400 μl | |

Fluorescent images were collected using Biorad's ChemiDoc system and processed with Image lab.
Giemsa staining of cells. As previously described⁵⁹.
Flow Cytometry Analyses for human and murine CD45. As previously described^{59, 60}. Antibodies used were monoclonal anti-human CD45 (clone HI30, cat. No 304016, Biolegend, 1:100) and monoclonal anti-mouse CD45 (Clone 30-F11, cat. No 1031066, Biolegend, 1:100).
Preparation and analysis of dNTP and NTP extracts: Cells were washed twice with ice-cold 1×PBS, and counted. To lyse cells, precipitate proteins and extract nucleotides and nucleosides, for each 5-10 million cells, 250 μL of 80% acetonitrile/water was added, and the cells were incubated on ice for 15 min. After incubation, the suspension was centrifuged at 140K rpm for 5 min. The supernatant from this first extraction was transferred to a clean tube. The remaining pellet was extracted again with fresh 80% acetonitrile/water, and supernatant from both extractions was combined, and then evaporated to dryness using a centrifugal evaporator. LCMS/MS: 1 mM Internal standards (13C9 15N3MP and d 13C9 15N3TP) solution (25 mM ammonia acetate, 10 mM DMHA, pH 8.0) was used to suspend Nucleotides and nucleosides extract. HPLC separation was carried on an ACQUITY UPLC HSS T3 Column, 100 Å, 1.8 μm, 2.1 mm×150 mm. A stepwise gradient program was applied with mobile phase A (25 mM Ammonia Bicarbonate, 10 mM DMHA, pH 8.0) and mobile phase B (60% Acetonitrile/water). The HPLC was interfaced with Thermofisher Quantiva triple quadruple mass spectrometer. The mass spectrometer was operated in MRM mode with optimized MRM transitions for each analyte. Data analysis: Xcalibur was used to process and quantify raw data. Briefly, a processing method was built using MRM transitions and peak retention times from standards. All samples were processed with the same method to generate integrated total ion intensity (integrated peak area) for each analyte. Manual inspection was performed to confirm the peak assignment and integration. The final report value was normalized to the internal standards and total number of cells used to generate the extract.
Treatment of a patient-derived xenotransplant model of treatment-resistant AML. Patient-derived primary AML cells from a patient with AML that had progressed on standard chemotherapy then decitabine salvage therapy, were transplanted by tail-vein injection (1.0×10⁶/mouse) into non-irradiated 6-8 week old NSG mice. Mice were anesthetized with isofluorane before transplantation. Mice were randomized to different treatments on Day 9 after inoculation, with treatments as indicated in each figure and legend. Doses of drugs used were: intra-peritoneal tetrahydrouridine (THU) 10 mg/kg given intra-peritoneal up to 3×/week; subcutaneous decitabine 0.2 mg/kg up to 3×/week (or 0.1 mg/kg when combined with THU); subcutaneous 5-azacytidine 2 mg/kg up to 3×/week (or 1 mg/kg when combined with THU); intra-peritoneal dT 2 g/kg up to 2×/week. Tail-vein blood samples for blood count measurement by HemaVet were obtained prior to leukemia inoculation, and at intervals thereafter as indicated in the figures. Mice were observed daily for signs of pain or distress, e.g., weight loss that exceeded 20% of initial total body weight, lethargy, vocalization, loss of motor function to any of their limbs, and were euthanized by an IACUC approved protocol if such signs were noted.
Bioinformatic and statistical analysis. Wilcoxon rank sum, Mann Whitney, and t tests were 2-sided unless otherwise stated because of apriori literature-based hypotheses (dCTP level analyses) and performed at the 0.05 significance level or lower (Bonferroni corrections were applied for instances of multiple parallel testing). Standard deviations (SD) and inter-quartile ranges (IQR) for each set of measurements were calculated and represented as y-axis error bars on each graph. Graph Prism (GraphPad, San Diego, Calif.) or SAS statistical software (SAS Institute Inc., Cary, N.C.) was used to perform statistical analysis including correlation analyses.

### Results

DNMT1 is not Depleted at Clinical Relapse or with In Vitro Resistance

Serial bone marrow biopsies from the same patient, before and during therapy with decitabine or 5-azacytidine, were cut onto the same glass slide and stained simultaneously to facilitate time-course comparison of DNMT1 protein levels quantified by immunohistochemistry and ImageIQ imaging/software (39 serial bone marrow samples from 13 patients, median treatment duration 372 days, range 170-1391) (FIG. 23A, B). At time-of-response, DNMT1 protein was markedly and significantly decreased by ˜50% compared to pre-treatment (FIG. 23B). At the time-of-relapse on-therapy, however, DNMT1 protein levels had rebounded to levels comparable to pre-treatment levels (FIG. 23B).

Since the pyrimidine metabolism enzymes DCK, UCK2, CDA and CAD are well-documented to mediate DNMT1-depleting ability of decitabine and 5-azacytidine (FIG. 23A), we used quantitative polymerase chain reaction (QRT-PCR) to measure expression of these enzymes in MDS patients' bone marrows pre-treatment and at relapse on-therapy with decitabine (n=13, median treatment-duration 175 days, range 97-922) or 5-azacytidine (n=14, median treatment-duration 433 days, range 61-1155) (FIG. 23C). DCK expression decreased by ˜50% at relapse on decitabine but increased by ˜8-fold at relapse on 5-azacytidine (FIG. 23C). Conversely, UCK2 expression increased by ˜8-fold at relapse on decitabine but decreased by ˜50% at relapse on 5-azacytidine (FIG. 23C). CDA expression increased ˜3-fold at relapse on decitabine and decreased by ˜50% at relapse on 5-azacytidine (FIG. 23C). CAD increased an average of ˜8-fold at relapse on either pro-drug, but not in all patients (FIG. 23C). The proliferation marker MK167 increased at relapse in almost all the patients, consistent with active progression of disease (FIG. 23C).

We then evaluated resistance to decitabine in vitro: AML cells (THP1, K562, OCI-AML3, MOLM13) were cultured in the presence of decitabine 0.5-1.5 μM (clinically relevant concentrations). After initial cytoreduction, AML cells proliferating exponentially through the decitabine emerged as early as 40 days after the first decitabine addition (FIG. 23D, 29). DNMT1 was not depleted from these decitabine-resistant AML cells despite the presence of decitabine (FIG. 23D). UCK2 and CDA protein levels were markedly elevated in decitabine-resistant vs vehicle-treated parental AML cells (FIG. 23D). Also upregulated was CAD, by total protein levels and by S1856 phosphorylation, a post-translational modification linked with its functional activation (FIG. 23D). In contrast, DCK protein levels were suppressed (FIG. 23D). In short, the in vitro resistance resembled the clinical resistance, with preserved DNMT1 and consistent pyrimidine metabolism enzyme expression changes.

### Decitabine and 5-Azacytidine Cause Deoxynucleotide Imbalances and Metabolic Compensations

We examined whether decitabine and 5-azacytidine cause deoxynucleotide imbalances, to potentially trigger compensatory responses from the pyrimidine metabolism network. AML cells (MOLM13, OCI-AML3, THP1) were treated with a single dose of vehicle, natural deoxycytidine 0.5 μM, decitabine 0.5 μM, natural cytidine 5 μM, or 5-azacytidine 5 μM in vitro, and effects on nucleotide levels and pyrimidine metabolism gene expression were measured 24 to 72 hours later (FIG. 24A).

Vehicle, deoxycytidine and cytidine did not impact proliferation of the AML cells (FIG. 24B). A single treatment with either decitabine or 5-azacytidine, on the other hand, significantly decreased AML cell proliferation (FIG. 24B). We then measured impact on dCTP and dTTP amounts at the 24 hour time-point: dCTP was significantly increased by decitabine but significantly decreased by 5-azacytidine (FIG. 24C). dTTP was decreased by both pro-drugs, but not to statistical significance (FIG. 24C). We then measured expression of key pyrimidine metabolism enzymes. Serial Western blot measurements of enzyme protein levels indicated peak expression changes occurred 48-96 hours after addition of pro-drug (FIG. 30). We thus focused repeat measurements, using both QRT-PCR and Western blots, at the 72 hour time-point. DCK mRNA and protein expression was significantly increased by 5-azacytidine but not decitabine treatment (FIG. 24D, E, 30). Conversely, UCK2 mRNA and protein expression was significantly increased by decitabine but not 5-azacytidine (FIG. 2D, E, 30). CDA mRNA and protein expression was significantly increased by both pro-drugs (FIG. 24D, E, 30). Neither pro-drug changed total CAD or cytidine triphosphate synthetase 1 (CTPS1) levels (CTPS1 executes a late step in de novo pyrimidine synthesis)(FIG. 24D, E, 30). Both pro-drugs did, however, decrease phosphorylation of CAD at serine 1856 (S1856)(FIG. 24E)—CAD S1856 phosphorylation is linked with functional upregulation of CAD-mediated de novo pyrimidine synthesis—thus, the pro-drugs acutely downregulated CAD function. Both decitabine and 5-azacytidine depleted DNMT1 as expected (FIG. 24E, 30).

We extended protein level analyses to additional pyrimidine metabolism enzymes playing nodal roles in nucleotide balance: thymidylate synthase (TYMS) is the major mediator of deoxythymidine triphosphate (dTTP) production. TYMS was downregulated by both pro-drugs, but to a noticeably greater extent by decitabine than 5-azacytidine (FIG. 24E, 30)—the natural substrate of TYMS is deoxyuridine monophosphate (dUMP), and decitabine and 5-azacytidine are metabolized into a dUMP analog Aza-dUMP by 2 vs 6 catalytic steps respectively (FIG. 30). 5-azacytidine, but not decitabine, decreased levels of the ribonucleotide reductase sub-unit RRM1 (FIG. 24E) (ribonucleotide reductase converts RNA molecules such as 5-azacytidine, after diphosphorylation, into DNA molecules such as decitabine), with less impact on the ribonucleotide reductase sub-unit RRM2A (FIG. 30).
DCK and UCK2 are Important for Maintaining dCTP and dTTP Respectively

To better understand contributions of DCK and UCK2 to dCTP and dTTP maintenance, we knocked DCK and UCK2 out of leukemia cells (HAP1) using CRISPR-Cas9 then measured levels of these nucleotides. DCK-knockout, but not UCK2-knockout, significantly decreased dCTP (FIG. 25A, B). Thus, DCK appears important to dCTP maintenance, consistent with DCK upregulation as an appropriate compensatory response to dCTP suppression by 5-azacytidine (FIG. 24). UCK2-knockout, but not DCK-knockout, significantly decreased dTTP (FIG. 25A, B). Thus, UCK2 appears important to dTTP maintenance, consistent with UCK2 upregulation as a response to dTTP suppression by decitabine (FIG. 24).

We also examined sensitivity of the DCK- and UCK2-knockout cells to decitabine and 5-azacytidine. DCK-knockout cells were relatively resistant to decitabine (concentrations for 50% growth inhibition [GI50] 12 vs 3 μM for parental cells), but more sensitive to 5-azacytidine (GI50 2 vs 4 μM for parental cells)(FIG. 25C). UCK2-knockout cells were relatively resistant to 5-azacytidine (GI50 15 μM vs 4 μM for parental cells), but more sensitive to decitabine (GI50 0.1 μM vs 3 μM for parental cells)(FIG. 25C).

### Resistance Countermeasures Evaluated In Vivo

We then examined solutions to resistance in a patient-derived xenotransplant (PDX) model of AML, derived from a patient with AML that was chemorefractory to both decitabine and cytarabine:

(a) Schedule decitabine administration to avoid DCK troughs: Immune-deficient mice were tail-vein innoculated with 1 million of these human AML cells each. On Day 9 after inoculation, mice were randomized to treatment with (i) vehicle; (ii) decitabine timed to avoid DCK troughs (Day 1 and Day 2 each week—Day 1, 2); or (iii) decitabine timed to coincide with DCK troughs (Day 1 and Day 4 each week—Day 1, 4) (FIG. 31A). Vehicle-treated mice showed distress on Day 45, at which point all mice were euthanized or sacrificed for analyses. The bone marrows of vehicle and Day 1, 4 treated mice, but not Day 1, 2 treated mice, were replaced by AML cells observed by light microscope (FIG. 31B) and by flow cytometry—human CD45+(hCD45+) AML cells were ˜92% with PBS, ˜63% with Day 1, 4 and ˜26% with Day 1, 2 treatment (FIG. 31C). Spleens were enlarged with effaced histology by AML with vehicle or Day 1, 4 treatment but had mostly preserved histology with Day 1, 2 treatment (FIG. 31D, E). Spleen weights as another measure of AML burden were also lowest with Day 1, 2 treatment (FIG. 31F).

These two schedules of decitabine administration were compared again but with waiting for signs of distress in individual mice rather than collective sacrifice at day 45 (FIG. 32A). Median survival (time-to-distress) was significantly better with Day 1, 2 (75 days) vs Day 1, 4 (60 days) or vehicle treatment (40 days) (FIG. 32B), and bone marrow and spleen AML burden was again lowest with Day 1, 2 vs Day 1, 4 or vehicle treatment (FIG. 32C-E).

Thus, scheduling decitabine administration to avoid DCK troughs (Day 1, 2) was superior to scheduling that coincided with these troughs (Day 1, 4).

(b) Combine with CDA and/or ribonucleotide reductase inhibitors: CDA can be inhibited by THU, while de novo pyrimidine synthesis can be inhibited at ribonucleotide reductase using deoxythymidine (dT) or hydroxyurea. NSG mice tail-vein innoculated with 1 million AML cells each were randomised to (i) vehicle; (ii) THU+dT, (iii) decitabine; (iv) THU+decitabine; or (v) THU+dT+decitabine (FIG. 26A). PBS and THU/dT-treated mice developed signs of distress, and were euthanized, on day 42. Mice receiving other treatments were sacrificed 3 weeks later (day 63) to increase chances of seeing differences in AML burden between these treatments (FIG. 26A). Visual inspection and flow cytometry demonstrated bone marrow replacement by human AML cells with vehicle and THU+dT treatment (>90% hCD45+), improved by decitabine alone (˜85% hCD45+) but most by THU+decitabine (˜35% hCD45+) and THU+dT+decitabine (˜42% hCD45+)(FIG. 26B, C)(that is, dT did not add further to the benefit from THU). Murine hematopoiesis was completely suppressed with vehicle and THU+dT (0% murine Cd45+), almost completely suppressed with decitabine-alone (˜5% mCd45+) but preserved with THU+decitabine (˜40% Cd45+) and THU+dT+decitabine (˜27% Cd45+)(FIG. 26C). Hemoglobin and platelets were most suppressed, and white cells (peripheral leukemia) most elevated, with vehicle or THU+dT treatment, but only mildly to moderately suppressed with any of the decitabine containing regimens (FIG. 26D). Spleen weights as a measure of AML burden were lowest with THU+decitabine- and THU+dT+decitabine (FIG. 26E). Spleen histology confirmed replacement by AML cells (with necrotic areas) with vehicle or THU+dT (FIG. 26F), decreased with decitabine-alone, and normal-appearance with THU+decitabine and THU+dT+decitabine (FIG. 26F). We also evaluated the use of hydroxyurea to inhibit ribonucleotide reductase: hydroxyurea 100 mg/kg IP was administered on Day 1 before THU+decitabine on days 2 and 3: hydroxyurea, like dT, did not add further benefit to THU+decitabine (FIG. 33A, B).

(c) Frequent, distributed vs pulse-cycled schedules of administration: DNMT1-depletion by decitabine or 5-azacytidine is S-phase dependent, suggesting frequent, distributed administration, to increase chances of overlap between malignant S-phase entries and drug exposures, could be better than pulse-cycled administration over a few consecutive days followed by weeks without therapy, designed for anti-metabolite/cytotoxic therapy that requires long treatment gaps to recover from toxic side-effects. Bone marrow AML burden was lowest with frequent-distributed administration of THU/decitabine 2×/week vs pulse-cycled administration for 5 days every 4 weeks (FIG. 33C). Vehicle-treated mice showed distress on Day 45, at which point all mice were euthanized or sacrificed for analyses. The bone marrows of vehicle treated mice demonstrated ˜95% replacement by human CD45+ AML cells. Bone marrow AML burden was decreased to ˜60% by pulse-cycled decitabine but decreased most to ˜10% with frequent-distributed decitabine, with inversely corresponding murine Cd45+ cells (FIG. 33D).

(d) Exploit cross-priming by Dec and 5Aza for each other: We compared head-to-head THU+decitabine 3×/week vs THU+5-azacytidine 3×/week and found no differences in efficacy between these two treatments (FIG. 27, 34). Then, since decitabine appears to cross-prime for 5-azacytidine activity by upregulating UCK2, while 5-azacytidine cross-primes for decitabine activity by upregulating DCK (FIG. 23-25, 30), we alternated THU+decitabine with THU+5-azacytidine week-to-week, and compared this to THU+decitabine or decitabine alone. Mice tail-vein innoculated with patient-derived AML cells (1×10⁶ cells/mouse) were randomized to (i) vehicle; (ii) decitabine alone; (iii) THU+decitabine; or (iv) THU+decitabine alternating with THU+5-azacytidine week-to-week (FIG. 28A). Median survival (time-to-distress) was best with THU+decitabine/THU+5-azacytidine (221 days) vs THU+decitabine (180 days), decitabine-alone (111 days) or vehicle (50 days) (FIG. 28B). Blood count stability during the weekly treatments was consistent with a non-cytotoxic mechanism-of-action of the therapies (shown also previously^{3, 26, 38-41}) (FIG. 28C). Eventual declines in hemoglobin and platelets were caused by progressive leukemia, shown by increasing peripheral leukemia cells (increasing white cell counts) (FIG. 28C), and by flow cytometry analyses of bone marrows harvested after euthanasia (FIG. 28D). Alternating THU+decitabine with THU+5-azacytidine in 4 week cycles, or simultaneous administration of THU+decitabine+5-azacytidine, did not add benefit over THU+decitabine or THU+5-azacytidine alone (FIG. 27, 34). Thus, the benefit of alternating THU+decitabine with THU+5-azacytidine depended on timing of alternation.

### Mechanisms-of-Resistance in Mice

Bone marrow cells harvested at day 63 when leukemia-inoculated mice were doing well on-therapy demonstrated DNMT1-depletion, with the greatest DNMT1-depletion with THU+decitabine alternating with THU+5-azacytidine week-to-week (˜65% DNMT1-depletion) vs THU+decitabine (˜50%), decitabine alone (˜35%) or vehicle (˜15%) (FIG. 28E). By contrast, bone marrow AML cells harvested at the time of progressive leukemia on these therapies demonstrated failure to deplete DNMT1 as measured by flow-cytometry (FIG. 28E). These bone marrows also demonstrated significant upregulations of CDA and CAD, with the greatest upregulations in AML cells from mice that received the alternating regimen and survived the longest (FIG. 28F).

Unbiased pre-clinical genetic studies have verified the central roles of DCK and UCK2 in determining sensitivity of leukemia cells to the pro-drugs decitabine and 5-azacytidine (companion manuscript). Here we found that malignant myeloid cells in vitro, in mice and in patients avoided DNMT1-depletion and resisted decitabine or 5-azacytidine via changes in expression of these and other key pyrimidine metabolism enzymes. We moreover found that these enzyme expression changes emerged from adaptive responses of the pyrimidine metabolism network, that senses and regulates deoxynucleotide amounts—decitabine and 5-azacytidine had opposite effects on dCTP amounts, via off-target depletion of TYMS (the major mediator of cellular dTTP production) and RRM1 (a key sub-unit of ribonucleotide reductase, the enzyme complex that converts ribonucleosides such as 5-azacytidine, after their diphosphorylation, into deoxyribonucleosides) respectively. TYMS, like DNMT1, methylates carbon #5 of the pyrimidine ring. This is the carbon that is substituted with a chemically active nitrogen in decitabine or 5-azacytidine, although in the case of TYMS, the substrate is deoxyuridine monophosphate (dUMP) instead of DNA-incorporated dCTP. A portion of administered decitabine, after phosphorylation by DCK then deamination by deoxycytidine deaminase (DCTD), is converted into a dUMP analog, Aza-dUMP. By depleting TYMS in this way, decitabine decreases dTTP that in turn increases dCTP, because dTTP inhibits ribonucleotide reductase-mediated reduction of CDP into dCDP. Decitabine inhibition of TYMS, and hence dTTP suppression/dCTP upregulation, has also been reported by others. A portion of administered 5-azacytidine can also be processed into Aza-dUMP, but via a more circuitous 6 instead of 2 catalytic steps. Instead, a more direct off-target action of 5-azacytidine, requiring only 2 catalytic steps to form Aza-CDP, is depletion of RRM1—off-target inhibition of ribonucleotide reductase by 5-azacytidine, and hence dCTP suppression, has also been reported by others. In brief, differential effects of 5-azacytidine and decitabine on RRM1 vs TYMS drive dCTP levels in opposite directions, triggering distinct responses from the pyrimidine metabolism network: DCK is particularly important for preserving dCTP, shown by a decrease in dCTP in DCK-knockout cells (shown also by others). Hence, upregulation of DCK is an appropriate adaptive response to dCTP suppression by 5-azacytidine. Rapid upregulation of DCK by 5-azacytidine has also been observed by others¹⁵. UCK2 on the other hand appears particularly important for dTTP maintenance, shown by a decrease in dTTP in UCK2-knockout cells. Therefore, UCK2 upregulation is an appropriate response to dTTP suppression by decitabine. CDA also contributes to dTTP maintenance (additional references below), thus CDA upregulation is another appropriate response to dTTP suppression by decitabine, again also observed by others. Cytarabine, another deoxycytidine analog that inhibits TYMS, has also been found to rapidly upregulate CDA in vitro and in the clinic, while hydroxyurea that inhibits ribonucleotide reductase does not (additional references).

This mode of resistance, that emerges adaptively from metabolic networks purposed toward homeostasis, does not require genetic mutations, and consistent with this, several studies that have looked for correlations between MDS/AML mutations and decitabine/5-azacytidine resistance have generated inconclusive or contradictory results. Expression levels of pyrimidine metabolism enzymes at baseline may also not necessarily be predictive^{52, 53}, since the metabolic reconfigurations are molded by treatment. We found that the consistent, predictable trajectory of the acute metabolic responses to the pro-drugs, however, facilitates outmaneuvering and even exploitation: (i) first, in PDX models of chemorefractory AML, scheduling decitabine administrations to avoid reactive troughs in DCK expression was notably superior to schedules that coincided with DCK troughs. (ii) Second, alternating decitabine with 5-azacytidine week-to-week, timed approximately to exploit priming of each pro-drug for activity of the other (UCK2 and DCK are maximally upregulated ˜96 hrs after decitabine and 5-azacytidine respectively), was significantly superior to administration of either pro-drug alone. The timing of alternation was important—alternating the pro-drugs in 4 week cycles, or their simultaneous administration, did not add benefit over the single agents. (iii) Third, frequent, distributed pro-drug administration, to increase possibilities of overlap between malignant cell S-phase entries and drug exposure windows, was superior to pulse-cycled administration schedules. Pulse-cycled schedules concentrate treatment over a few consecutive days separated by multi-week intervals, recovery periods necessary with cytotoxic treatments—such long gaps are not needed if decitabine or 5-azacytidine doses are selected for non-cytotoxic DNMT1-depletion, as shown also in previous clinical trials. Observations from others support rationalization of treatment schedules to increase S-phase dependent DNMT1-depletion: RNA-sequencing analysis of patients' baseline bone marrows found that a gene expression signature of low cell cycle fraction predicted non-response to pulse-cycled 5-azacytidine therapy, and regulatory approval of decitabine and 5-azacytidine to treat myeloid malignancies occurred after doses were lowered from initially evaluated, toxic high doses, then administered more frequently¹. (iv) Fourth, adding THU, to inhibit the catabolic enzyme CDA that severely limits decitabine and 5-azacytidine tissue-distribution and half-lives, and that is rapidly upregulated by decitabine (and to a lesser extent 5-azacytidine) in vitro and in vivo, also extended decitabine or 5-azacytidine anti-AML efficacy in vivo. An important detail in such combinations was that the decitabine and 5-azacytidine doses were lowered to preserve a non-cytotoxic DNMT1-targeting mode of action. Stated another way, dose-escalations of decitabine or 5-azacytidine are not a solution for resistance since this compromises therapeutic-index: AML cells indefinitely self-replicate/proliferate and therefore have the opportunity to be educated for resistance from repeated treatment exposures, but normal myelopoiesis proliferates and terminally differentiates in successive waves, each treatment naïve and vulnerable to cytotoxic effects of high doses.

CAD, the enzyme that initiates de novo pyrimidine synthesis, was downregulated acutely by decitabine or 5-azacytidine, but was upregulated at stable resistance, the only discrepancy we found between acute vs chronic metabolic reconfiguration. This discrepancy may reflect the terminal-differentiation induced acutely but not at stable resistance. We did not find benefit in vivo from combining decitabine with dT or hydroxyurea to inhibit ribonucleotide reductase. Others, however, have found promise in vitro combining 5-azacytidine with other inhibitors of de novo pyrimidine synthesis: pyrazofurin to inhibit CTPS1⁵⁵, PALA to inhibit CAD¹⁷ or leflunomide to inhibit DHODH⁵⁶. As per combinations with CDA-inhibitors, 5-azacytidine or decitabine combinations with de novo synthesis inhibitors will likely require reductions in 5-azacytidine/decitabine doses to preserve therapeutic index, since toxicities caused failure of previous clinical trials of high dose 5-azacytidine and pyrazofurin⁵⁷.

In sum, we found that resistance to decitabine and 5-azacytidine emerges from adaptive responses of the pyrimidine metabolism network. These network responses can be anticipated and exploited using simple and practical treatment modifications that preserve the vital therapeutic index of non-cytotoxic DNMT1-depletion.

### REFERENCES

1. Saunthararajah Y. Key clinical observations after 5-azacytidine and decitabine treatment of myelodysplastic syndromes suggest practical solutions for better outcomes. Hematology Am Soc Hematol Educ Program 2013; 2013: 511-521.
2. Vesely J, Cihak A, Sorm F. Characteristics of mouse leukemic cells resistant to 5-azacytidine and 5-aza-2′-deoxycytidine. Cancer Res 1968 October; 28(10): 1995-2000.
3. Ng K P, Ebrahem Q, Negrotto S, Mahfouz R Z, Link K A, Hu Z, et al. p53 independent epigenetic-differentiation treatment in xenotransplant models of acute myeloid leukemia. Leukemia 2011 November; 25(11): 1739-1750.
4. Saunthararajah Y, Sekeres M, Advani A, Mahfouz R, Durkin L, Radivoyevitch T, et al. Evaluation of noncytotoxic DNMT1-depleting therapy in patients with myelodysplastic syndromes. J Clin Invest 2015 Mar. 2; 125(3): 1043-1055.
5. Tsai H C, Li H, Van Neste L, Cai Y, Robert C, Rassool F V, et al. Transient Low Doses of DNA-Demethylating Agents Exert Durable Antitumor Effects on Hematological and Epithelial Tumor Cells. Cancer Cell 2012 Mar. 20; 21(3): 430-446.
6. Negrotto S, Ng K P, Jankowska A M, Bodo J, Gopalan B, Guinta K, et al. CpG methylation patterns and decitabine treatment response in acute myeloid leukemia cells and normal hematopoietic precursors. Leukemia 2012 February; 26(2): 244-254.
7. Momparler R L, Cote S, Momparler L F. Epigenetic action of decitabine (5-aza-2′-deoxycytidine) is more effective against acute myeloid leukemia than cytotoxic action of cytarabine (ARA-C). Leuk Res 2013 May 6.
8. Trowbridge J J, Sinha A U, Zhu N, Li M, Armstrong S A, Orkin S H. Haploinsufficiency of Dnmt1 impairs leukemia stem cell function through derepression of bivalent chromatin domains. Genes Dev 2012 Feb. 15; 26(4): 344-349.
9. Milhem M, Mahmud N, Lavelle D, Araki H, DeSimone J, Saunthararajah Y, et al. Modification of hematopoietic stem cell fate by 5aza 2′ deoxycytidine and trichostatin A. Blood 2004 Jun. 1; 103(11): 4102-4110.
10. Hu Z, Negrotto S, Gu X, Mahfouz R, Ng K P, Ebrahem Q, et al. Decitabine maintains hematopoietic precursor self-renewal by preventing repression of stem cell genes by a differentiation-inducing stimulus. Mol Cancer Ther 2010 June; 9(6): 1536-1543.
11. Chaurasia P, Gajzer D C, Schaniel C, D'Souza S, Hoffman R. Epigenetic reprogramming induces the expansion of cord blood stem cells. J Clin Invest 2014 Apr. 24.
12. Suzuki M, Harashima A, Okochi A, Yamamoto M, Nakamura S, Motoda R, et al. 5-Azacytidine supports the long-term repopulating activity of cord blood CD34(+) cells. AmJHematol 2004; 77(3): 313-315.
13. Velcheti V, Schrump D, Saunthararajah Y. Ultimate Precision: Targeting Cancer but Not Normal Self-replication. American Society of Clinical Oncology educational book American Society of Clinical Oncology Annual Meeting 2018 May 23; (38): 950-963.
14. Stegmann A P, Honders W H, Willemze R, Ruiz vHV, Landegent J E. Transfection of wild-type deoxycytidine kinase (dck) cDNA into an AraC- and DAC-resistant rat leukemic cell line of clonal origin fully restores drug sensitivity. Blood 1995; 85(5): 1188-1194.
15. Antonsson B E, Avramis V I, Nyce J, Holcenberg J S. Effect of 5-azacytidine and congeners on DNA methylation and expression of deoxycytidine kinase in the human lymphoid cell lines CCRF/CEM/0 and CCRF/CEM/dCk-1. Cancer Res 1987; 47(14): 3672-3678.
16. Qin T, Jelinek J, Si J, Shu J, Issa J P. Mechanisms of resistance to 5-aza-2′-deoxycytidine in human cancer cell lines. Blood 2009; 113(3): 659-667.
17. Grant S, Bhalla K, Gleyzer M. Effect of uridine on response of 5-azacytidine-resistant human leukemic cells to inhibitors of de novo pyrimidine synthesis. Cancer Res 1984 Dec; 44(12 Pt 1): 5505-5510.
18. Liacouras A S, Anderson E P. Uridine-cytidine kinase. IV. Kinetics of the competition between 5-azacytidine and the two natural substrates. Molecular pharmacology 1979 March; 15(2): 331-340.
19. Sripayap P, Nagai T, Uesawa M, Kobayashi H, Tsukahara T, Ohmine K, et al. Mechanisms of resistance to azacitidine in human leukemia cell lines. Exp Hematol 2014 April; 42(4): 294-306 e292.
20. Qin T, Castoro R, El Ahdab S, Jelinek J, Wang X, Si J, et al. Mechanisms of resistance to decitabine in the myelodysplastic syndrome. PLoS One 2011; 6(8): e23372.
21. Wu P, Geng S, Weng J, Deng C, Lu Z, Luo C, et al. The hENTI and DCK genes underlie the decitabine response in patients with myelodysplastic syndrome. Leuk Res 2015 February; 39(2): 216-220.
22. Camiener G W, Smith C G. Studies of the enzymatic deamination of cytosine arabinoside. I. Enzyme distribution and species specificity. BiochemPharmacol 1965; 14(10): 1405-1416.
23. Zauri M, Berridge G, Thezenas M L, Pugh K M, Goldin R, Kessler B M, et al. CDA directs metabolism of epigenetic nucleosides revealing a therapeutic window in cancer. Nature 2015 Aug. 6; 524(7563): 114-118.
24. Beausejour C M, Eliopoulos N, Momparler L, Le N L, Momparler R L. Selection of drug-resistant transduced cells with cytosine nucleoside analogs using the human cytidine deaminase gene. Cancer Gene Ther 2001; 8(9): 669-676.
25. Eliopoulos N, Cournoyer D, Momparler R L. Drug resistance to 5-aza-2′-deoxycytidine, 2′,2′-difluorodeoxycytidine, and cytosine arabinoside conferred by retroviral-mediated transfer of human cytidine deaminase cDNA into murine cells. Cancer Chemother Pharmacol 1998; 42(5): 373-378.
26. Ebrahem Q, Mahfouz R, Ng K P, Saunthararajah Y. High cytidine deaminase expression in the liver provides sanctuary for cancer cells from decitabine treatment effects. Oncotarget 2012 October; 3(10): 1137-1145.
27. Liu Z, Marcucci G, Byrd J C, Grever M, Xiao J, Chan K K. Characterization of decomposition products and preclinical and low dose clinical pharmacokinetics of decitabine (5-aza-2′-deoxycytidine) by a new liquid chromatography/tandem mass spectrometry quantification method. Rapid CommunMass Spectrom 2006; 20(7): 1117-1126.
28. Mahfouz R Z, Jankowska A, Ebrahem Q, Gu X, Visconte V, Tabarroki A, et al. Increased CDA expression/activity in males contributes to decreased cytidine analog half-life and likely contributes to worse outcomes with 5-azacytidine or decitabine therapy. Clin Cancer Res 2013 Feb. 15; 19(4): 938-948.
29. Mahfouz R Z, Koh L S, Teo M, Chee C L, Toh H C, Saunthararajah Y. Gender, cytidine deaminase, and 5-aza/decitabine—response. Clin Cancer Res 2013 Jun. 1; 19(11): 3106-3107.
30. DeZern A E, Zeidan A M, Barnard J, Hand W, Al Ali N, Brown F, et al. Differential response to hypomethylating agents based on sex: a report on behalf of the MDS Clinical Research Consortium (MDS CRC). Leukemia & lymphoma 2017 June; 58(6): 1325-1331.
31. Grant S, Bhalla K, Gleyzer M. Interaction of deoxycytidine and deoxycytidine analogs in normal and leukemic human myeloid progenitor cells. LeukRes 1986; 10(9): 1139-1146.
32. Ng S K, Rogers J, Sanwal B D. Alterations in differentiation and pyrimidine pathway enzymes in 5-azacytidine resistant variants of a myoblast line. J Cell Physiol 1977 February; 90(2): 361-347.
33. Lane A N, Fan T W. Regulation of mammalian nucleotide metabolism and biosynthesis. Nucleic Acids Res 2015 Feb. 27; 43(4): 2466-2485.
34. Almqvist H, Axelsson H, Jafari R, Dan C, Mateus A, Haraldsson M, et al. CETSA screening identifies known and novel thymidylate synthase inhibitors and slow intracellular activation of 5-fluorouracil. Nat Commun 2016; 7: 11040.
35. Heinemann V, Plunkett W. Modulation of deoxynucleotide metabolism by the deoxycytidylate deaminase inhibitor 3,4,5,6-tetrahydrodeoxyuridine. Biochemical pharmacology 1989 Nov. 15; 38(22): 4115-4121.
36. Bianchi V, Pontis E, Reichard P. Regulation of pyrimidine deoxyribonucleotide metabolism by substrate cycles in dCMP deaminase-deficient V79 hamster cells. Mol Cell Biol 1987 Dec; 7(12): 4218-4224.
37. Nathanson D A, Armijo A L, Tom M, Li Z, Dimitrova E, Austin W R, et al. Co-targeting of convergent nucleotide biosynthetic pathways for leukemia eradication. J Exp Med 2014 Mar. 10; 211(3): 473-486.
38. Gu X, Ebrahem Q, Mahfouz R Z, Hasipek M, Enane F, Radivoyevitch T, et al. Leukemogenic nucleophosmin mutation disrupts the transcription factor hub that regulates granulomonocytic fates. J Clin Invest 2018 Oct. 1; 128(10): 4260-4279.
39. Saunthararajah Y, Hillery C A, Lavelle D, Molokie R, Dom L, Bressler L, et al. Effects of 5-aza-2′-deoxycytidine on fetal hemoglobin levels, red cell adhesion, and hematopoietic differentiation in patients with sickle cell disease. Blood 2003 Dec 1; 102(12): 3865-3870.
40. Molokie R, Lavelle D, Gowhari M, Pacini M, Krauz L, Hassan J, et al. Oral tetrahydrouridine and decitabine for non-cytotoxic epigenetic gene regulation in sickle cell disease: A randomized phase 1 study. PLoS medicine 2017 September; 14(9): e1002382.
41. Lavelle D, Vaitkus K, Ling Y, Ruiz M A, Mahfouz R, Ng K P, et al. Effects of tetrahydrouridine on pharmacokinetics and pharmacodynamics of oral decitabine. Blood 2012 Feb. 2; 119(5): 1240-1247.
42. Aimiuwu J, Wang H, Chen P, Xie Z, Wang J, Liu S, et al. RNA-dependent inhibition of ribonucleotide reductase is a major pathway for 5-azacytidine activity in acute myeloid leukemia. Blood 2012 May 31; 119(22): 5229-5238.
43. Austin W R, Armijo A L, Campbell D O, Singh A S, Hsieh T, Nathanson D, et al. Nucleoside salvage pathway kinases regulate hematopoiesis by linking nucleotide metabolism with replication stress. J Exp Med 2012 Nov. 19; 209(12): 2215-2228.
44. Momparler R L, Laliberte J. Induction of cytidine deaminase in HL-60 myeloid leukemic cells by 5-aza-2′-deoxycytidine. LeukRes 1990; 14(9): 751-754.
45. Mameri H, Bieche I, Meseure D, Marangoni E, Buhagiar-Labarchede G, Nicolas A, et al. Cytidine Deaminase Deficiency Reveals New Therapeutic Opportunities against Cancer. Clin Cancer Res 2017 Apr. 15; 23(8): 2116-2126.
46. Im A P, Sehgal A R, Carroll M P, Smith B D, Tefferi A, Johnson D E, et al. DNMT3A and IDH mutations in acute myeloid leukemia and other myeloid malignancies: associations with prognosis and potential treatment strategies. Leukemia 2014 September; 28(9): 1774-1783.
47. DiNardo C D, Patel K P, Garcia-Manero G, Luthra R, Pierce S, Borthakur G, et al. Lack of association of IDH1, IDH2 and DNMT3A mutations with outcome in older patients with acute myeloid leukemia treated with hypomethylating agents. Leukemia & lymphoma 2014 August; 55(8): 1925-1929.
48. Traina F, Visconte V, Elson P, Tabarroki A, Jankowska A M, Hasrouni E, et al. Impact of molecular mutations on treatment response to DNMT inhibitors in myelodysplasia and related neoplasms. Leukemia 2014 January; 28(1): 78-87.
49. Metzeler K H, Walker A, Geyer S, Garzon R, Klisovic R B, Bloomfield C D, et al. DNMT3A mutations and response to the hypomethylating agent decitabine in acute myeloid leukemia. Leukemia 2012 May; 26(5): 1106-1107.
50. Bejar R, Lord A, Stevenson K, Bar-Natan M, Perez-Ladaga A, Zaneveld J, et al. TET2 mutations predict response to hypomethylating agents in myelodysplastic syndrome patients. Blood 2014 Oct. 23; 124(17): 2705-2712.
51. Braun T, Itzykson R, Renneville A, de Renzis B, Dreyfus F, Laribi K, et al. Molecular predictors of response to decitabine in advanced chronic myelomonocytic leukemia: a phase 2 trial. Blood 2011 Oct. 6; 118(14): 3824-3831.
52. Unnikrishnan A, Papaemmanuil E, Beck D, Deshpande N P, Verma A, Kumari A, et al. Integrative Genomics Identifies the Molecular Basis of Resistance to Azacitidine Therapy in Myelodysplastic Syndromes. Cell reports 2017 Jul. 18; 20(3): 572-585.
53. Valencia A, Masala E, Rossi A, Martino A, Sanna A, Buchi F, et al. Expression of nucleoside-metabolizing enzymes in myelodysplastic syndromes and modulation of response to azacitidine. Leukemia 2014 March; 28(3): 621-628.
54. Awada H, Mahfouz R Z, Kishtagari A, Kuzmanovic T, Durrani J, Kerr C M, et al. Extended experience with a non-cytotoxic DNMT1-targeting regimen of decitabine to treat myeloid malignancies. Br J Haematol 2019 Nov. 17.
55. Cadman E, Eiferman F, Heimer R, Davis L. Pyrazofurin enhancement of 5-azacytidine antitumor activity in L5178Y and human leukemia cells. Cancer Res 1978 Dec; 38(12): 4610-4617.
56. Imanishi S, Takahashi R, Katagiri S, Kobayashi C, Umezu T, Ohyashiki K, et al. Teriflunomide restores 5-azacytidine sensitivity via activation of pyrimidine salvage in 5-azacytidine-resistant leukemia cells. Oncotarget 2017 Sep. 19; 8(41): 69906-69915.
57. Martelo O J, Broun G O, Jr., Petruska P J. Phase I study of pyrazofurin and 5-azacytidine in refractory adult acute leukemia. Cancer treatment reports 1981 March-April; 65(3-4): 237-239.
58. Wang H, Chen P, Wang J, Santhanam R, Aimiuwu J, Saradhi U V, et al. In vivo quantification of active decitabine-triphosphate metabolite: a novel pharmacoanalytical endpoint for optimization of hypomethylating therapy in acute myeloid leukemia. The AAPS journal 2013 January; 15(1): 242-249.
59. Gu X, Hu Z, Ebrahem Q, Crabb J S, Mahfouz R Z, Radivoyevitch T, et al. Runxl regulation of Pu.1 corepressor/coactivator exchange identifies specific molecular targets for leukemia differentiation therapy. J Biol Chem 2014 May 23; 289(21): 14881-14895.
60. Hu Z, Gu X, Baraoidan K, Ibanez V, Sharma A, Kadkol S, et al. RUNX1 regulates corepressor interactions of PU.1. Blood 2011 Jun. 16; 117(24): 6498-6508.

### ADDITIONAL REFERENCES

1. de Saint Vincent B R, Dechamps M, Buttin G. The modulation of the thymidine triphosphate pool of Chinese hamster cells by dCMP deaminase and UDP reductase. Thymidine auxotrophy induced by CTP in dCMP deaminase-deficient lines. J Biol Chem 1980 Jan. 10; 255(1): 162-167.
2. Moro-Bulnes A, Castillo-Acosta V M, Valente M, Carrero-Lerida J, Perez-Moreno G, Ruiz-Perez L M, et al. Contribution of Cytidine Deaminase to Thymidylate Biosynthesis in Trypanosoma brucei: Intracellular Localization and Properties of the Enzyme. mSphere 2019 Aug. 7; 4(4).
3. Neuhard J. Pyrimidine nucleotide metabolism and pathways of thymidine triphosphate biosynthesis in Salmonella typhimurium. Journal of bacteriology 1968 November; 96(5): 1519-1527.
4. Andersen L, Kilstrup M, Neuhard J. Pyrimidine, purine and nitrogen control of cytosine deaminase synthesis in Escherichia coli K 12. Involvement of the glnLG and purR genes in the regulation of codA expression. Archives of microbiology 1989; 152(2): 115-118.
5. Momparler R L, Laliberte J. Induction of cytidine deaminase in HL-60 myeloid leukemic cells by 5-aza-2′-deoxycytidine. LeukRes 1990; 14(9): 751-754.
6. Mameri H, Bieche I, Meseure D, Marangoni E, Buhagiar-Labarchede G, Nicolas A, et al. Cytidine Deaminase Deficiency Reveals New Therapeutic Opportunities against Cancer. Clin Cancer Res 2017 Apr. 15; 23(8): 2116-2126.
7. Meyers R, Malathi V G, Cox R P, Silber R. Studies on nucleoside deaminase. Increase in activity in HeLa cell cultures caused by cytosine arabinoside. J Biol Chem 1973 Sep. 10; 248(17): 5909-5913.
8. Chen P, Aimiuwu J, Xie Z, Wei X, Liu S, Klisovic R, et al. Biochemical modulation of aracytidine (Ara-C) effects by GTI-2040, a ribonucleotide reductase inhibitor, in K562 human leukemia cells. The AAPS journal 2011 March; 13(1): 131-140.
9. Steuart C D, Burke P J. Cytidine deaminase and the development of resistance to arabinosyl cytosine. NatNew Biol 1971; 233(38): 109-110.
10. Roberts D, Loehr E V. Depression of thymidylate synthetase activity in response to cytosine arabinoside. Cancer Res 1972 June; 32(6): 1160-1169.
11. International Patent Publication WO2017/096357.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

We claim:

1. A method of treating a human subject with cancer comprising, or consisting essentially of:
a) administering 5-15 mg/kg of a cytidine deaminase inhibitor to a human subject at a first time, wherein said human subject has cancer;
b) administering 0.07-0.4 mg/kg of decitabine to said subject at a second time, wherein said second time is between 0.5 and 7 hours after said first time;
c) administering 5-15 mg/kg of a cytidine deaminase inhibitor to said subject at a third time, wherein said third time is between 60 and 105 hours, or 106-240 hours, after said first time;
d) administering 0.5-4 mg/kg of 5-azacytidine to said subject at a fourth time, wherein said fourth time is: i) 0.5 and 7 hours after said third time, and ii) between 60 and 105 hours, or 106-240 hours, after said second time; and
e) repeating steps a)-d) at least once, wherein said administering in step a) is conducted between 60 and 105 hours, or 106-240 hours, after said fourth time.

2. The method of claim 1, wherein said subject does not receive any decitabine or 5-azacytidine except at the times specified in steps b) and d) respectively.

3. The method of claim 1, wherein said repeating steps a)-d) at least once comprises repeating steps a)-d) at last five times.

4. The method of claim 1, wherein said repeating steps a)-d) at least once comprises repeating steps a)-d) at last twelve times.

5. The method of claim 1, wherein said cancer is selected from the group consisting of pancreatic cancer, a myeloid cancers, a lymphoid cancers, and small cell lung cancer.

6. The method of claim 1, wherein said administering 5-15 mg/kg of said cytidine deaminase inhibitor in steps a) and c) comprises administering 9-11 mg/kg of said cytidine deaminase inhibitor.

7. The method of claim 1, wherein said administering 0.07-0.4 mg/kg of decitabine comprises administering 0.1-0.3 mg/kg of said decitabine.

8. The method of claim 1, wherein said administering 0.07-0.4 mg/kg of decitabine comprises administering 0.15-0.17 mg/kg of said decitabine.

9. The method of claim 1, wherein said administering 0.5-4 mg/kg of 5-azacytidine comprises administering 1-3 mg/kg of said 5-azacytidine.

10. The method of claim 1, wherein said administering 0.5-4 mg/kg of 5-azacytidine comprises administering 1.5-1.7 mg/kg of said 5-azacytidine.

11. The method of claim 1, wherein said between 0.5 and 7 hours in step b) is between 1 and 5 hours.

12. The method of claim 1, wherein said between 0.5 and 7 hours in step d) is between 1 and 5 hours.

13. The method of claim 1, wherein said between 60 and 105 hours in step d) is between 70 and 98 hours.

14. The method of claim 1, wherein said between 106 and 240 hours in step c) is between 160 and 170 hours.

15. The method of claim 1, further comprising: administering said subject a composition comprising human granulocyte colony-stimulating factor (GCSF).

16. The method of claim 1, further comprising: testing a sample from said subject to determine absolute neutrophil count (ANC).

17. The method of claim 16, wherein said ANC is below 1.5×10⁹, and said method further comprises administering said subject a composition comprising human granulocyte colony-stimulating factor (GCSF).

18. The method of claim 1, wherein said subject does not receive any cytidine deaminase inhibitor except at the times specified in steps a) and c).

19. The method of claim 1, wherein said cytidine deaminase inhibitor comprises tetrahydrouridine.

20. A method of treating a human subject with cancer comprising, or consisting essentially of:
a) administering decitabine to said subject on day one;
b) administering decitabine to said subject on day two, wherein day two is the day immediately after said day one;
c) administering decitabine to said subject on day eight, wherein said day eight is seven days after said day one, and wherein said subject does not receive any decitabine between said administering in step b) and said administering in step c); and
d) administering decitabine to said subject on day nine, wherein said day nine is eight days after said day one.

21. The method of claim 20, further comprising: repeating steps c) and d) at least once, wherein said subject does not receive any decitabine expect during the repeated administration of steps c) and d).

22. The method of claim 21, wherein steps c) and d) are repeated at least twice.

23. The method of claim 20, further comprising administering a cytidine deaminase inhibitor to said subject on the same days as when said decitabine is administered.

24. The method of claim 23, wherein said cytidine deaminase inhibitor is administered at a dosage of 5-15 mg/kg.

25. The method of claim 20, wherein each administration of said decitabine is administered at dosage of 0.07-0.4 mg/kg.


SEQUENCE LISTING (10 sequences)

SEQ ID NO: 1 - DNA, 22 bp, Artificial sequence
  DNA Primer
ctgacttctacactgagcatgg

SEQ ID NO: 2 - DNA, 22 bp, Artificial sequence
  DNA Primer
cacgcattgacaggttaatcac

SEQ ID NO: 3 - DNA, 21 bp, Artificial sequence
  DNA Primer
aagggtacaaggatttcaggg

SEQ ID NO: 4 - DNA, 21 bp, Artificial sequence
  DNA Primer
acaatatacgtaccatccggc

SEQ ID NO: 5 - DNA, 18 bp, Artificial sequence
  DNA Primer
aagctgcccgtctttctc

SEQ ID NO: 6 - DNA, 21 bp, Artificial sequence
  DNA Primer
accacttcccaatcttcacac

SEQ ID NO: 7 - DNA, 21 bp, Artificial sequence
  DNA Primer
atccccgtgtatgactttgtc

SEQ ID NO: 8 - DNA, 21 bp, Artificial sequence
  DNA Primer
cttcatctggaacaggtctcg

SEQ ID NO: 9 - DNA, 19 bp, Artificial sequence
  DNA Primer
acatcgctcagacaccatg

SEQ ID NO: 10 - DNA, 22 bp, Artificial sequence
  DNA Primer
tgtagttgaggtcaatgaaggg